(19) United States
(12) Patent Application Publication
(10) Pub. No.: US 2026/0007805 A1
(43) Pub. Date: Jan. 8, 2026

(54) COMPOSITIONS AND METHODS FOR TREATING DIABETES

(71) Applicant: ASPECT BIOSYSTEMS LTD., Vancouver (CA)

(72) Inventors: Tamer Mohamed, Vancouver (CA); Sam Wadsworth, Vancouver (CA); Simon Beyer, Richmond (CA); Spiro Getsios, Nanaimo (CA); Valerio Russo, Vancouver (CA); Sheng Pan, Richmond (CA); Navid Hakimi, Vancouver (CA); Mohammad Mostafa Kamal Khan, Vancouver (CA); Yang Yu, Delta (CA); Reza Barada Jalili, Vancouver (CA)

(21) Appl. No.: 18/866,498

(22) PCT Filed: May 15, 2023
(86) PCT No.: PCT/CA23/50668

§ 371 Date: Nov. 15, 2024

Related U.S. Application Data
(60) Provisional application No. 63/342,120, filed on May 15, 2022.

(51) Int. Cl. A61L 27/38; A61K 9/00; A61K 35/39; A61K 47/36; A61L 27/20; A61L 27/52; A61L 27/54; A61P 3/10

(52) U.S. Cl. CPC A61L 27/3804; A61K 9/0024; A61K 35/39; A61K 47/36; A61L 27/20; A61L 27/3882; A61L 27/52; A61L 27/54; A61P 3/10; A61L 2300/64

(57) ABSTRACT

Aspects of the disclosure include an implantable composition/medical device encapsulating insulin-producing cells for use in the treatment and management of diabetes, generally comprising a multilayer lattice structure fabricated from a continuously bioprinted cell-laden core/shell fiber, the lattice structure having a defined infill density and at least coating applied to the lattice post-printing, and preferably at least one conformal coating. In this way, multilayer lattice structures are provided with improved stability, retrievability, and functionality.

### CROSS-REFERENCE TO RELATED APPLICATIONS

This application claims priority to U.S. Provisional Patent Application No. 63/342,120, filed May 15, 2022, the contents of which are incorporated herein by reference in their entirety.

### FIELD OF DISCLOSURE

The present disclosure relates generally to implantable therapeutic devices for the treatment and management of diseases, disorders, or conditions. Specifically, the invention relates to three-dimensional biological structures comprising pancreatic islet cells printed from digital files, and compositions and methods of use thereof, for use in the treatment of diabetes in subjects in need thereof.

### BACKGROUND OF THE DISCLOSURE

Type 1 diabetes (T1D) is an autoimmune disease in which the immune system destroys the insulin-producing pancreatic β-cells. Blood glucose (BG) can be managed via daily injections or infusion of exogenous insulin, yet even with close monitoring and adjustment of insulin dosage, such an approach does not cure the disease nor prevent many adverse effects associated with diabetes. For example, hyper- and hypoglycemia are common side effects of exogenous insulin therapy that can lead to, inter alia, irreversible tissue and organ damage.

Type 2 diabetes (T2D) is a different disease etiology, usually occurring later in life, but with similar loss of blood glucose control and resulting organ damage. The majority of T2D patients can control their blood glucose levels through modifications in diet and lifestyle, however many T2D patients become less responsive to insulin which often leads to failure of pancreatic function, and as a result around 30% of T2D patients are also dependent on exogenous insulin to maintain normoglycemia.

Recent clinical trial results have demonstrated the promise of pancreatic islet transplantation as a potential curative therapy. Butler and Gale, J Clin Invest. (2022) 132(3):e158305. doi.org/10.1172/JCI158305. In particular, proprietary embryonic stem cell-derived islet cells including functional β cells were generated using a differentiation protocol established by Melton and colleagues at Harvard University (Pagliuca et al. Cell (2014); 159(2):428-439), and the first patient to receive these cells in an ongoing clinical trial showed a remarkable clinical effect. (Vertex announces positive day 90 data for the first patient in the phase 1/2 clinical trial dosed with VX-880, a novel investigational stem cell-derived therapy for the treatment of type 1 diabetes. News release. Oct. 18, 2021). Unfortunately, however, this methodology requires the use of lifelong immunosuppressive therapy to counteract the powerful host immune response triggered against the transplanted cells, which can lead to serious side effects (Weir G C and Bonner-Weir S, Diabetes. (1997); 46: 1247-1256; Hafiz M M et al., Transplantation. (2005); 80:1718-1728; Niclauss N et al., Transplantation. (2011); 91: 714-722).

Cell encapsulation has been persistently explored as an alternative approach for transplanting islets or stem cell-derived beta-like cells without immunosuppression (Calafiore and Basta, Adv Drug Deliv Rev. (2014); 67-68: 84-92). Yet despite decades of research across the globe, an effective clinical application of cell encapsulation remains elusive (Orive et al., Trends Pharmacol Sci. (2015); 36:537-546). While planar diffusion chambers such as the Viacyte device showed great promise in small animal studies, Nat Biotechnol. 2014; 32:929, they faced considerable challenges in the scale up to human patients due to their intrinsically low surface area for mass transfer. Calafiore, supra. Moreover, a clinical trial of a modified device called VC01-103 was terminated due to “insufficient functional product engraftment,” suggesting that the recovered devices were coated with fibrous tissue. Henry et al. Diabetes. 2018; 67(Suppl 1):138-OR. Portals permitting vascular ingrowth were then added to the device but also conferred access to the immune system, which then made induction and maintenance immunosuppressive therapy necessary. Shapiro et al. Cell Rep Med. 2021; 2(12):100466

Still missing, then, is a cell encapsulation device that can protect the encapsulated cells and/or tissue fragments from the host's immune system without hindering the passage of nutrients, oxygen, and secreted products (e.g., insulin), have sufficient strength and elasticity to survive in vivo for prolonged periods of time, and be readily retrievable when depleted or compromised. These requirements are further complicated by the fact that implantation of such devices into the body can trigger an orchestrated biological response by both innate and adaptive immune systems referred to as a foreign body response (FBR). This response is mediated in part by macrophages that overexpress extracellular matrix (ECM) proteins such as fibronectin, and produce pro-fibrogenic factors that enhance fibrogenesis by fibroblasts, resulting in the formation of a fibrotic capsule around the device. This fibrous capsule can interfere with device function, particularly when they contain therapeutic cell populations requiring access to nutrients and oxygen flow and efflux of therapeutic proteins in order to perform their intended function.

A wide range of materials from naturally occurring polymers to synthetic materials have been described as generating fibrotic responses. Ward W K, J Diabetes Sci Technol. (2008); 2: 768-777; Zhang L et al., Nature Biotech. (2013); 31: 553-556; Ratner B D, Journal of Controlled Release. (2002); 78: 211-218. Furthermore, physical parameters such as the shape, size, and texture of the synthetic tissue structures are intrinsic properties that also contribute to FBR. The surface of a synthetic tissue structure can affect the behavior of macrophages and other immune cells, with structures having a smooth surface generally inducing less inflammation. Mariani E et al., Int J Mol Sci. (2019); 20: doi: 10.3390/ijms20030636. Changes in surface roughness at the nanoscale have been associated with increased protein adsorption (Hulander M et al., Int J Nanomedicine. (2011); 6: 2653-2666; Roach P., J Mater Sci Mater Med. (2007); 18: 1263-1277; Scopelliti P E et al., PLOS ONE. (2010); 5: e11862; and different nanostructured topographies can affect cellular interactions (Baker D W et al., Biomacromolecules. (2011); 12: 997-1005; Jahed Z., Biomaterials. (2014); 35: 9363-9371).

Accordingly, in order to fully realize the clinical potential of encapsulated cells in the treatment of T1D, improvements in both design and materials are needed to accommodate the opposing objectives of immune protection and nutrient passage, and to help mitigate the FBR response. There is a need for synthetic tissue structures that reduce or avoid FBR and immune system recognition while ensuring adequate passage of oxygen and nutrients to cells. There is also a need for synthetic structures and methods of generation thereof, in which patterning is consistent and reliable, and realized via effective synthetic tissue fiber-to-fiber adhesion.

### SUMMARY OF DISCLOSURE

The present invention successfully resolves the foregoing conflicting objectives in the art with an implantable medical device for the treatment of diabetes, comprising a lattice structure comprising a continuously bioprinted core/shell fiber encapsulating pancreatic islet cells, wherein the lattice structure further comprises at least one coating imparting effective fiber-to-fiber (F-F) adhesion to enhance the structural stability and retrievability of the device. In embodiments, the lattice structure comprises at least one conformal coating as described herein, which may impart the lattice structure with anti-FBR properties. In embodiments, the lattice structure is fabricated with a defined infill density which enables effective diffusion properties and vascular in-growth resulting in the effective and sustained delivery of oxygen and nutrients to the pancreatic islet cells of the lattice structure. The pancreatic islet cells in turn exhibit improved survival and functionality, and release insulin in vivo as a function of blood glucose levels, to effectively treat type 1 diabetes in subjects in need thereof.

Aspects of the invention include an implantable composition/medical device for the treatment of type 1 diabetes, comprising a multilayer lattice structure comprising a continuously bioprinted core/shell fiber encapsulating a plurality of pancreatic islet cells, and at least one coating surrounding said multilayer lattice structure, wherein the multilayer lattice structure has an infill density of between about 10% and about 90%, or between about 20% and about 80%, or between about 30% and about 70%, or between about 40% and about 60%; and preferably between about 50% and about 70%, or between about 55% and about 65%, or about 60%.

In embodiments, said lattice structure has an infill density of about 30%, about 40%, about 50%, about 60%, or about 70%, or about 80%.

In embodiments, the multilayer lattice structure comprises at least one conformal coating.

In embodiments, the continuously bioprinted core/shell fiber comprises a solid core and at least one shell, optionally wherein the solid core has a material strength less than that of the shell.

In embodiments, the coating comprises a hydrogel having a material strength less than both the core and the at least one shell of the fiber.

In embodiments, the solid core, the at least one shell, and the coating comprise the same hydrogel material; preferably wherein the hydrogel material is alginate.

In embodiments, the solid core, the at least one shell, and/or the coating comprises a chemically modified alginate.

In embodiments, the solid core comprises between about 1.2 to about 1.8% alginate, preferably about 1.5% alginate.

In embodiments, the at least one shell comprises between about 1.4% to about 3.0% alginate; preferably between about 1.5% to about 2.5% alginate; more preferably between about 1.8% and about 2.2% alginate.

In embodiments, the coating comprises between about 0.2% alginate to about 2% alginate, or between about 0.25% alginate to about 1.5% alginate; preferably between about 0.3% to about 1.0% alginate; more preferably between about 0.4% to about 0.8% alginate.

In embodiments, said at least one coating comprises a first inner coating and a second outer coating. In embodiments, the first inner coating comprises a hydrogel having a material strength greater than that of the second outer coating. In embodiments, the first inner coating comprises between about 1.4% to about 3.0% alginate, preferably between about 1.5% to about 2.5% alginate, more preferably between about 1.8% and about 2.2% alginate. In embodiments, the second outer coat comprises between about 0.25% alginate to about 1.5% alginate, preferably between about 0.3% alginate to about 1.0% alginate, more preferably between about 0.4% alginate to about 0.8% alginate.

In embodiments, the pancreatic islet cells are human pancreatic islet cells, optionally wherein the pancreatic islet cells comprise re-aggregated islets. In embodiments, the pancreatic islet cells are stem cell-derived human pancreatic islet cells, optionally wherein the stem cell-derived pancreatic islet cells comprise clusters of cells

In embodiments, the lattice structure comprises at least two, three, four, or five layers formed by the continuous fiber, preferably wherein the lattice structure comprises two layers or three layers or four layers, more preferably wherein the lattice structure comprises four layers.

In embodiments, a diameter of the continuous fiber is between about 0.2-2.0 mm, or between about 0.5-1.5 mm, between about 0.5-0.9 mm, or between about 900 μm to about 1200 μm, preferably wherein the diameter is between about 950 μm to about 1100 μm.

In embodiments, the solid core has a diameter between about 500 μm and about 800 μm, preferably between about 600 μm and about 700 μm, more preferably about 650 μm.

In embodiments, the at least one shell has a thickness of between about 50 μm and about 125 μm, preferably between about 75 μm and about 100 μm.

In embodiments, the coating has a thickness of between about 50 μm to about 125 μm, preferably between about 75 μm and about 100 μm.

In embodiments, the solid core and/or the at least one shell is compartmentalized along the length of the fiber. The pancreatic islet cells may be encapsulated in the core and/or the at least one shell. In embodiments, the plurality of pancreatic islet cells are encapsulated in the solid core. In embodiments, the plurality of pancreatic islet cells are encapsulated in the at least one shell

Aspects of the invention include a method of treating a diabetic subject, comprising implanting a composition/medical device of the present disclosure into the diabetic subject. In embodiments, the diabetic subject is a human subject suffering from Type 1 diabetes.

In embodiments, said implanting is carried out via a laparoscopic procedure.

In embodiments, the method further comprises retrieving the composition/medical device from the subject after 1-12 months, 18 months, 24 months, 2 years, 3 years, 4 years, 6 years, 8 years, or 10 years following implantation of the composition/medical device.

In embodiments, the method further comprises implanting another composition/medical device of the present disclosure into said subject following said retrieving step.

Aspects of the invention include a method of fabricating an implantable composition/medical device of the present disclosure, comprising providing a bioprinting system comprising a fabrication platform for supporting a continuously bioprinted fiber during printing, patterning, and/or processing, the fabrication platform comprising a frame defining a void and comprising a plurality of posts on opposing sides of the frame for securing and suspending the continuously bioprinted fiber during printing; a print head comprising a plurality of microfluidic channels to selectively provide a respective plurality of materials to a dispensing orifice; a positioning unit for positioning the frame in three dimensional space with respect to the print head; and at least one dispensing means for dispensing the fiber from the dispensing orifice; via the bioprinting system, dispensing the fiber around a plurality of said posts to form lattice structure comprising at least two, three, four, or five layers of the fiber; and

coating the lattice structure with at least one conformal coat after printing is completed.

In embodiments, said fabrication platform is submerged in a cross-linker bath during dispensing of said continuously bioprinted fiber.

In embodiments, said fabrication platform comprising said lattice structure is submerged into a cross-linker bath following dispensing of said continuously bioprinted fiber.

In embodiments, said at least one coating step comprises submerging the fabrication platform comprising the lattice structure in at least one coating solution.

In embodiments, said at least one coating step comprises dispensing at least one coating solution onto the lattice structure via the dispensing orifice following dispensing of said continuously bioprinted fiber.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A depicts an illustrative example of an implantable multilayer lattice structure of the present disclosure.

FIG. 1B depicts an illustrative example of an implantable multilayer lattice structure from another angle, and depicts a cross-sectional view of the lattice structure.

FIG. 1C depicts another illustrative example of an implantable multilayer lattice structure.

FIG. 1D depicts exemplary lattice structures with varying infill densities.

FIG. 2 depicts an exemplary means for suspending a bioprinted tissue fiber of the present disclosure during printing, patterning, and/or processing thereof.

FIGS. 3A-3B depict illustrations of segmented/compartmentalized continuous fibers of the lattice structures according to the present disclosure.

FIG. 4 depicts a general process flow for the fabrication of multilayer lattice structures of the present disclosure.

FIG. 5A is a schematic representation and bright field image of bioprinted primary human islet tissue.

FIG. 5B illustrates live/dead staining of bioprinted primary islets.

FIG. 5C depicts data from a Glucose-Stimulated Insulin Secretion (GSIS) assay performed using primary human and rat islets.

FIG. 6A illustrates random-fed blood glucose measurements following streptozotocin (STZ) treatment and intraperitoneal (IP) implantation of bioprinted human islet tissue in NSG (NOD scid gamma) mice over 80 days.

FIG. 6B illustrates human C-peptide levels measured in mouse plasma over 80 days using ELISA.

FIG. 6C illustrates data from an oral glucose tolerance test (OGTT) performed at day 80 to assess kinetics of blood glucose normalization following a fasting period and subsequent glucose challenge in NSG mice with bioprinted islet tissues or healthy, non-STZ treated control mice.

FIG. 7A illustrates blood glucose measurements following omental pouch implantation of bioprinted rat islet tissue in STZ-treated nude rats (n=2) over 180 days.

FIG. 7B shows H&E (high and low magnification) and insulin (islets) or CD31 (endothelial cells) immunohistochemistry (IHC) performed on sections from fixed, bioprinted tissue explanted at 180 days.

FIG. 8A illustrates blood glucose measurements following omental pouch implantation of bioprinted Lewis rat islet tissue in STZ-treated Sprague-Dawley (SD) rats over 90 days.

FIG. 8B shows H&E and insulin (islets) or CD31 (endothelial cells) IHC performed on sections from fixed, bioprinted tissue explanted at 60 days.

FIG. 9A is a schematic illustration of a biomanufacturing process of the present disclosure.

FIG. 9B shows bioprinted pancreatic tissue used for studies in rats compared to scaled-up tissue for large animals.

FIG. 9C shows viability of bioprinted neonatal porcine islets confirmed up to 14 days post-print.

FIG. 10 depicts a process flow to manufacture implantable lattice structures containing bioprocessed pancreatic islets in materials that protect the allogeneic cells from host immune cell attack.

FIG. 11A is a schematic of a 10×10 mm lattice structure of the present disclosure.

FIG. 11B is an image of a coated 10×10 mm lattice structure coupled to frame of the present disclosure.

FIGS. 11C-11D depict images of the coated 10×10 mm lattice structures of FIGS. 11A-11B uncoupled from the frame.

FIG. 12A depicts live/dead staining of a coated 10×10 mm lattice structure as compared to a coated 18×18 mm lattice structure each comprising HepG2 aggregates, assessed at 0 days following printing.

FIG. 12B depicts live/dead the coated 10×10 mm lattice structure as compared to a coated 18×18 mm fiber structure each comprising HepG2 aggregates, assessed at 5 days following printing.

FIGS. 13A-13B show images of a 10×10 mm lattice structure after coating, on frame (FIG. 13A), and uncoupled from frame (FIG. 13B).

FIG. 14 summarizes stability data for coated 10×10 mm lattice structures comprising HepG2 aggregates or primary rat islets (PRI).

FIGS. 15A-15C depict images of three coated 10×10 mm lattice structures with HA-containing cores.

FIGS. 15D-15F depict images of three coated 10×10 mm lattice structures without HA-containing cores (i.e., SLG100 but not HA).

FIG. 16A illustrates live/dead staining of coated 10×10 mm lattice structures loaded with PRIs, assessed at 0 days post printing.

FIG. 16B illustrates live/dead staining of the coated 10×10 mm lattice structures of FIG. 16A, assessed at 3 days post printing.

FIG. 17 is a table illustrating stability data of lattice structures printed on a frame of the present disclosure and then coated as compared to in lieu of such a frame.

FIGS. 18A-18C are images of lattice structures printed on a frame of the present disclosure and then coated, following their being subjected to a stability test, the data of which is summarized at FIG. 17.

FIGS. 18D-18F are images of lattice structures printed in lieu of a frame of the present disclosure and then coated, following their being subjected to a stability test, the data of which is summarized at FIG. 17.

FIG. 19A is a microscopic image of a coated lattice structure printed by way of a frame of the present disclosure.

FIGS. 19B-19C are microscopic images of coated lattice structures printed in lieu of a frame of the present disclosure.

FIG. 20 is an image depicting uncoated inside-out cross-linked bioprinted fiber structures printed in lieu of a frame of the present disclosure.

FIG. 21 depicts a fiber structure with both a first inner conformal coating and a second outer conformal coating.

FIGS. 22A-22C are microscopic images of a lattice structure with or without cells based on core-switching to compartmentalize the fiber structure.

### DETAILED DESCRIPTION

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

The term “hydrogel” as used herein refers to a composition comprising water and a network or lattice of polymer chains that are hydrophilic.

The term “sheath fluid” or “sheath solution” as used herein refers to a fluid that is used, at least in part, to envelope or “sheath” a material as the material is passing through a fluid channel. In some embodiments, a sheath fluid comprises an aqueous solvent, e.g., water or glycerol. In some embodiments, a sheath fluid comprises a chemical cross-linking agent. Non-limiting examples of cross-linking agents include divalent cations (e.g. Ca²⁺, Ba²⁺, Sr²⁺, etc.), thrombin, and pH modifying chemicals, such as sodium bicarbonate.

The terms “segmented/compartmentalized” as used herein refer to a discontinuous nature of a type of material and/or a biological material included in core or shell(s) of the fibers disclosed herein, e.g. wherein there are intentional gaps in the deposition of the type of material and/or biological material along a length of the fiber. The spacing (e.g., length) between such segments/compartments may be regular (e.g., an approximately same spacing between regions of biological material), or the spacing may be different.

The term “solid core” as used herein refers to a core of a fiber of the present disclosure that is comprised of a particular material (e.g., hydrogel cross-linkable by a chemical cross-linking agent), such that the core does not comprise a lumen along the entire length of the fiber. The term is not intended to refer to a core that is entirely impenetrable along its length, as solid cores of the present disclosure may enable the passage of particular fluids, molecules and/or ionic species throughout the core.

The term “biocompatible materials” as used herein refers to materials in which biological materials including but not limited to cells can be incorporated into and/or be in contact with said biocompatible materials, and where said biocompatible materials do not exhibit an adverse effect on the ability of the biological materials to carry out one or more functions (e.g., cellular functions including but not limited to secretion of biologically relevant molecular species, agonist/receptor binding, signal transduction, and the like).

The term “immunoprotective” as used herein refers broadly to a design aspect of a fiber of the present disclosure that serves to reduce, prevent or eliminate the host immune response including, e.g., immune cell invasion of the fiber upon implantation of the fiber into a body (e.g., mammalian body).

The term “agent” as used herein refers to any protein, nucleic acid molecule (including chemically modified nucleic acid molecules), antibody, small molecule, organic compound, inorganic compound, or other molecule of interest. An agent can include a biologically relevant agent, a therapeutic agent, a diagnostic agent, a pharmaceutical agent, a chelating agent, a cross-linking agent, etc. A therapeutic or pharmaceutical agent is one that alone or together with an additional compound induces a desired response (such as inducing a therapeutic or prophylactic effect when administered in a manner consistent with the present disclosure to a subject. A biologically relevant agent is one that supports another biological process, for example an agent that supports cell viability.

### Introduction

Diabetes mellitus (i.e., diabetes) is a disease in which the body's ability to produce or respond to the hormone insulin is impaired, resulting in abnormal metabolism of carbohydrates and elevated levels of glucose in the blood and urine. The disease is subdivided into several sub-types, described alternatively as Type 1 diabetes mellitus, insulin-dependent diabetes mellitus (IDDM), mature onset diabetes of the young (MODY), latent adult diabetes (LADA), brittle diabetes, lean diabetes, Type 1.5, Type 2, Type 3, obesity-related diabetes, gestational diabetes, and other nomenclature accepted by the field.

In general, a subject with insulin-dependent diabetes is required to administer exogenous insulin to sufficiently lower blood glucose. A subject with insulin-dependent diabetes may benefit from a cell replacement therapy in which insulin-producing cells are implanted to the subject whether that disease is labeled as Type 1, MODY, LADA, brittle, lean, Type 1.5, Type 2, Type 3, obesity related diabetes or any combination thereof.

However, as mentioned above, there are numerous issues hindering the practical realization of cell replacement therapy, including islet survival and a requirement that patients be subjected to long-term immunosuppressive drug therapy. Cell encapsulation strategies have the potential to mitigate such issues, yet in turn are hampered by other issues including but not limited to FBR to implanted devices.

3D bioprinting is an additive manufacturing process where synthetic tissue structures, optionally cell laden, are laid down in a layer-by-layer fashion to obtain multi-layer 3D structures. Various types of 3D bioprinting techniques have been developed, including extrusion (Panwar A et al., Molecules. (2016); 21: 685; Sakai S et al., Biofabrication. (2018); 10: 045007; Han H W and Hsu S H, Neural Regener. Res. (2017); 12: 1595), inkjet (Gao G et al., Biotechnol. Lett. (2015); 37: 2349; Gao G and Cui X, Biotechnol. Lett. (2016); 38: 203; Bsoul A et al., Lab Chip. (2016); 16: 3351) laser assisted (Sorkio A et al., Biomaterials. (2018); 171: 57; Pagès E et al., J. Nanotechnol. Eng. Med. (2015); 6: 021006; Catros S et al., In Vivo and In Situ Biofabrication by Laser-Assisted Bioprinting, Elsevier, Winston-Salem, USA. (2015)), and stereolithographic (SLA) (Miri A K et al., Adv. Mater. (2018); 30: 1800242; Wang Z et al., ACS Appl. Mater. Interfaces. (2018); 10; 26859; Wang Z et al., Biofabrication. (2015); 7: 045009) printing methods. Of these, extrusion is one of the most common, whereby bioinks are dispensed through one or more syringes to form layer-by-layer scaffolds from fibers.

Advances have also led to the use of microfluidics-based 3D bioprinting systems (Beyer S T et al., in 2013 Transducers Eurosensors XXVII 17^{th} Int. Conf. Solid-State Sensors, Actuators, Microsystems. IEEE, Piscataway, NJ (2013); pp. 1206-1209; Beyer S T et al., in The 17^{th} Int. Conf. on Miniaturized Systems for Chemistry and Life Sciences. (2013); pp. 176-178). With these systems and techniques, a plurality of materials (e.g., bioink, cross-linker, etc.) flow through microchannels which can allow for precision control of one or more of flow, switching, mixing, and the like. When used with a sheath flow that surrounds at least one inner material, microfluidic bioprinting can reduce shear stress during the printing process. Microfluidics-based 3D bioprinting can also advantageously allow for the intersecting of material flows as they exit independent flow paths into a single flowpath (e.g., dispensing channel), to facilitate the production of structures having a core surrounded by one or more shells.

Despite the promise of 3D bioprinting strategies, however, issues including but not limited to immune protection, adequate oxygen and nutrient passage, and avoidance of FBR remain technical problems in need of solution. The inventors herein have recognized the above-mentioned issues, and have developed compositions and methods to address them.

### Multilayer Lattice Structure for the Treatment of Diabetes

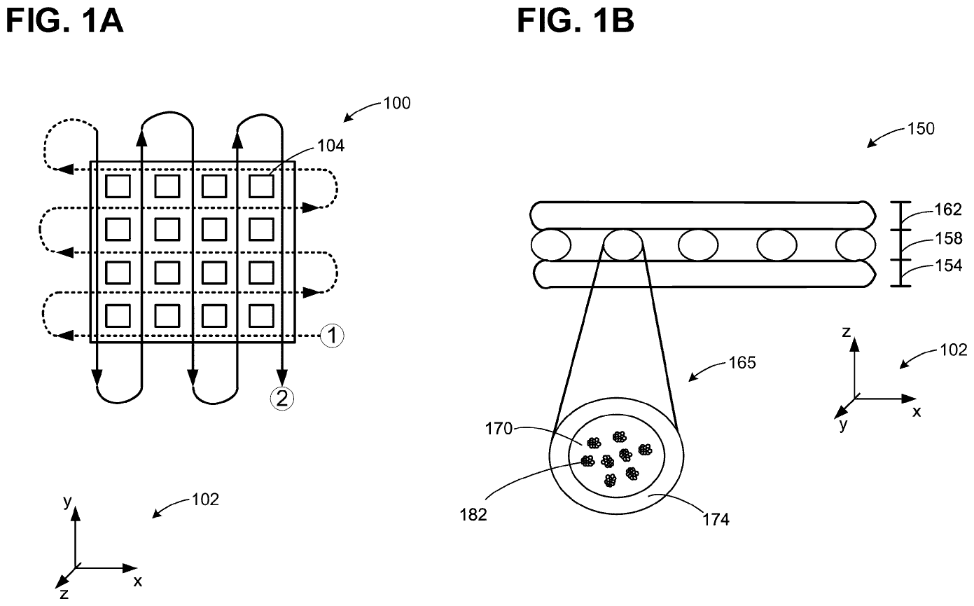

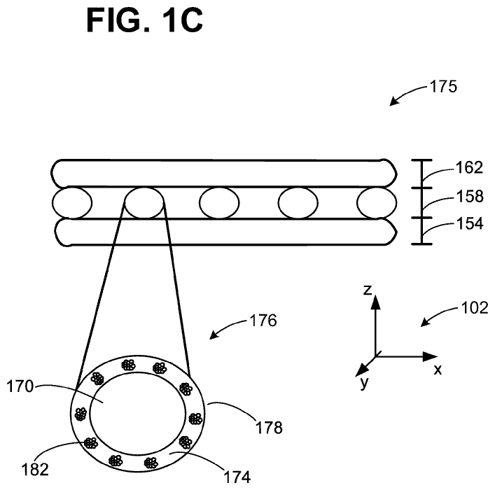

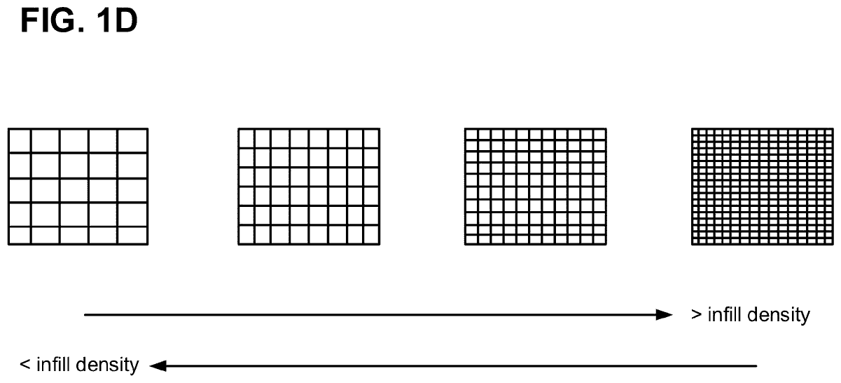

One aspect the present invention is directed to an implantable multilayer lattice structure for the treatment of diabetes (e.g., Type 1 diabetes). Turning to FIG. 1A, depicted is an exemplary illustration of such a lattice structure 100. Lattice structure 100 at FIG. 1A is viewed along the z-axis (see Cartesian coordinate system 102 at FIG. 1A). In embodiments, the lattice structure 100 can take the form of a grid arrangement, with regions 104 of open space. In embodiments, a single continuous fiber can be bioprinted into a grid arrangement, and then coated (coating not shown at FIG. 1A) to form the final multilayer lattice structure 100. For example, starting from point (1) at FIG. 1A, a first layer can be formed in the manner indicated by dashed arrows, followed by laying down a second layer on top of the first layer as indicated by solid arrows, ending at point (2). In this way, the multilayer lattice structure can be formed from a single continuous fiber, and can then be coated in its entirety. While FIG. 1A is discussed with regard to two layers, a multilayer lattice structure formed from a single continuous fiber can comprise more than two layers, for example 3, 4, 5, 6, 7, 8, 9, or 10 layers, or even greater than 10 layers in some examples. It is to be understood that the depiction at FIG. 1A is meant to be illustrative, and non-limiting.

Lattice structure 100 is shown as approximately square, although structures of the present disclosure need not necessarily be square but can be comprised of other shapes including but not limited to rectangular, oval, hexagonal, circular, and the like. Dimensions of the lattice structure comprise those suitable for implantation into a particular animal (e.g., human, dog, cat, rat, pig, etc.). In embodiments where the lattice structure is approximately square, dimensions of the structure may be between about 8 mm×8 mm to about 150 mm×150 mm. In embodiments where the lattice structure is of a different shape than square, area of the lattice structure can range from between about 64 mm² to about 22500 mm², or any value there between.

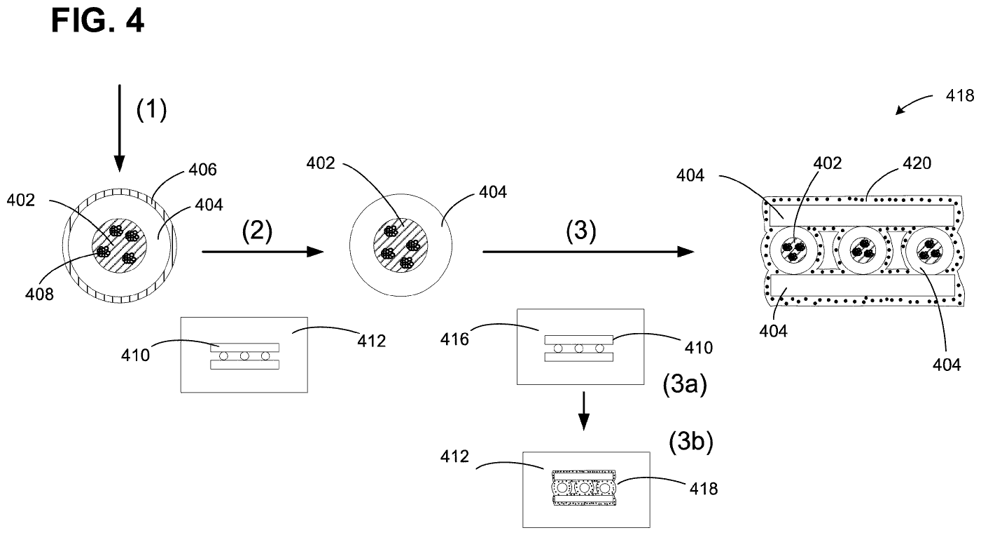

Turning now to FIG. 1B, depicted is a multilayer lattice structure 150 which is substantially the same as that of lattice structure 100 of FIG. 1A, but is shown along the y-axis (see Cartesian coordinate system 102 at FIG. 1B), and is depicted as having three layers, including first layer 154, second layer 158, and third layer 162. In embodiments, the multilayer lattice structure 150 is formed in similar fashion as discussed above, including forming a grid arrangement from one continuous fiber, and then uniformly applying a coating (coating not shown at FIG. 1B, but see FIG. 4) to produce the final lattice structure 150. As shown in inset 165, fibers 163 of the finished lattice structure will comprise a solid core 170, at least one shell 174, and at least one coating (not shown at FIG. 1B) over the exposed fiber surfaces in the lattice structure. In embodiments, solid core 170 encapsulates the pancreatic islets 182, as depicted at FIG. 1B. In embodiments, shell 174 encapsulates the pancreatic islets (see lattice structure 175 and corresponding inset 176 at FIG. 1C, coating not shown at FIG. 1C). Although fibers of FIGS. 1B-1C illustrate a single shell, alternative embodiments comprising more than one shell, and/or optionally more than one coating are contemplated. For example a multilayer lattice structure of the present disclosure can include 1, 2, 3, 4, or 5 shells, and 1, 2, 3, 4, or 5 coatings. Bioprinted fibers themselves can also be coated in some examples.

In embodiments, the solid core, at least one shell and one or more coatings are comprised of biocompatible material(s). Examples of biocompatible materials relevant to the present disclosure can include but are not limited to alginate, collagen, decellularized extracellular matrices, hyaluronic acid (HA), polyethylene glycol (PEG), fibrin, gelatin, gelatin methacrylate (GEL-MA), silk, chitosan, cellulose, polycaprolactone (PCL), poly(lactic acid) (PLA), poly(oligoethylene glycol methacrylate) (POEGMA), or a combination thereof. In embodiments, the solid core, at least one shell and one or more coatings are comprised of a hydrogel material. Hydrogel materials relevant to the present disclosure can include but are not limited to alginate (e.g., SLG-100 alginate), chitosan, GEL-MA, agarose, PEG, PCL, poly-L-lysine (PLL), triazole, fucoidan, poly(ethylene glycol) diacrylate (PEGDA), poly(-ethylene glycol)-tetra-acrylate (PEGTA), poly (vinyl alcohol) (PVA), Hyaluronic acid (HA), hyaluronic acid methacryloyl (HAMA), collagen, methacrylated collagen (ColMA), gelatin, gellan, fibrin (fibrinogen), and combinations thereof. In some embodiments, HA can be used to enhance/increase viscosity.

In embodiments, the diameter of the solid core of a continuously bioprinted fiber of the present disclosure is between about 500 μm and about 800 μm, preferably between about 600 μm and about 700 μm, for example about 600 μm, 610 μm, 620 μm, 630 μm, 640 μm, 650 μm, 660 μm, 670 μm, 680 μm, 690 μm, 700 μm, or any value there between. In some embodiments, the diameter of the solid core is about 650 μm.

In embodiments, the thickness of the at least one shell of a continuously bioprinted fiber of the present disclosure is between about 50 μm and about 125 μm, preferably between about 75 μm and about 100 μm, for example about 75 μm, 80 μm, 85 μm, 90 μm, 95 μm, 100 μm, or any value there between.

In embodiments, each layer of the multilayer lattice structure (e.g., lattice structure 150 at FIG. 1B, lattice structure 175 at FIG. 1C) has a thickness between about 500 μm to about 1500 μm, preferably wherein the thickness of each layer is between about 800 μm to about 1200 μm, more preferably wherein the thickness of each layer is between about 950 μm to about 1100 μm, for example about 950 μm, 975 μm, 1000 μm, 1025 μm, 1050 μm, 1075 μm, 1100 μm, or any value there between.

In embodiments, a thickness of a coating applied to a multilayer lattice structure of the present disclosure is between about 50 μm and about 125 μm, preferably between about 75 μm and about 100 μm, for example about 75 μm, 80 μm, 85 μm, 90 μm, 95 μm, 100 μm, or any value there between.

In embodiments, the core and/or shell of a continuously bioprinted fiber of the present disclosure comprises a plurality of pancreatic islets (see e.g., FIG. 1B-1C). Discussed herein, an “islet equivalent” (IEQ) refers to an islet of a particular diameter, containing an approximate amount of cells. Devices of the present disclosure may include some appropriate number of IEQs, depending on the species.

### Infill Density

The infill density of the lattice structures of the present disclosure represents an important parameter determining diffusional flow as well as vascularization. For example, lattice structures with too high an infill density may impede/degrade diffusional flow through and/or host tissue ingrowth. Alternatively, lattice structures with too low of an infill density may be undesirable in terms of one or more of structural stability, F-F adhesion, ease of retrievability, and the like. Infill density, as discussed herein, is referred to as percent infill density of the 3D lattice structures including the one or more coating(s). Turning to FIG. 1D, depicted are exemplary lattice structures of varying infill densities for reference, with the infill density of the depicted lattice structures increasing from left to right. A lattice structure having a completely filled fibrous structure (i.e., no spaces) would thus correspond to an infill density of 100%, whereas a lattice structure that is 90% unoccupied by any fibrous structure corresponds to an infill density of 10%.

In embodiments, the infill density of suitable multilayer lattice structures of the present disclosure is between about 10% and about 90%, for example between about 20% and about 80%, for example between about 30% and about 70%, for example between about 40% and about 60%, about 30%, about 40%, about 50%, about 60%, about 70%, or about 80%, or any value there between. Preferably, the infill density is between about 50% and about 60%.

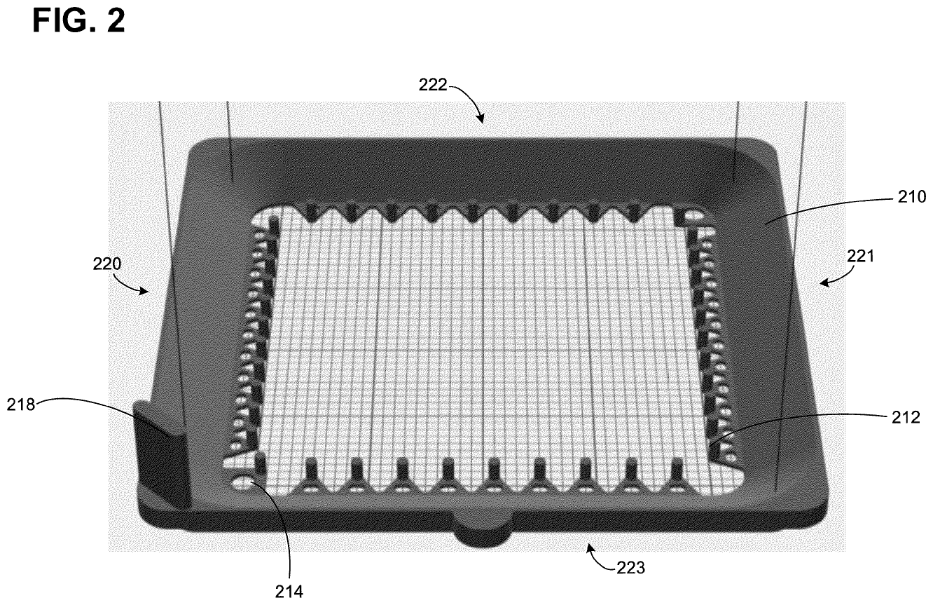

For multilayer lattice structures of the present disclosure, infill density is advantageously controlled via the manner in which the structures are fabricated. Specifically, as explained in more detail below, and as detailed in co-pending provisional application 63/342,118, a continuous fiber can be precisely printed into a lattice of 2, 3, 4, 5, or more layers using the fabrication platform described therein. Briefly, FIG. 2 illustrates an exemplary embodiment of such a platform, comprising a frame 210 comprising posts 212. To generate the lattice, a continuous fiber can be bioprinted around a plurality of posts 212, for example in the manner depicted and discussed with regard to FIG. 1A. The posts 212 serve to impart tension to the fiber as it is being printed, which advantageously maintains the fiber in each row/column a uniform distance away from the fiber corresponding to adjacent row/column in terms of each layer, and ensuring linearity of the fiber between each opposing post as the lattice is being fabricated. Following the fabrication of the lattice structure, one or more conformal coatings can be applied to the entirety of the exposed surfaces of the lattice structure, without disrupting the structure, and even prior to (or during) the effective fiber-to-fiber adhesion of the overall printed structure. As such these conformal coatings can be advantageously used to impart stability and/or impart anti-FBR properties to a tissue fiber structure Handle 218 may be used to transport/store frame 210 and corresponding attached lattice structure and/or to perform post-printing processing steps, e.g. one or more coatings, while the lattice structure remains attached to the frame. In this way, the fabrication platform can be advantageously used during printing, patterning and/or post-printing processing of the devices of the present disclosure.

In embodiments, the infill density may be a function of a number of posts on the fabrication platform and their relationship to one another (e.g., distance from adjacent posts and/or number of posts on opposing and/or adjacent sides of the frame). For example, FIG. 2 depicts frame 210 which is substantially square, and includes a greater number of posts along side 220 and side 221 (11 posts on each side), and a lesser number of posts (9 posts on each side) along side 222 and side 223. In other embodiments, all sides of such a frame may include an equal number of posts. In either case, embodiments preferably include at least two corner posts 214 to assist in transitioning the fiber the layers altering.

### Material Strengths

In embodiments, the solid core, the at least one shell, and the one or more coating(s) may comprise the same or different material strengths. For example and without limitation, for a multilayer lattice structure that is comprised of a solid core, at least one shell, and at least one coating, the core may have a first material strength, the at least one shell may have a second material strength, and the at least one coating may have a third material strength. In such an example, each of the first, second, and third material strength may be the same. In other embodiments, the first material strength, second material strength, and third material strength may be different, or may be different than the other two. In embodiments where the continuous fiber comprises two or more shells, each shell may comprise a different material strength. Additionally or alternatively, in some embodiments where a lattice structure is comprised of at least two coatings, each coating may comprise a different material strength. In an exemplary embodiment, a multilayer lattice structure is comprised of a solid core, a shell, and a coating, wherein a material strength of the shell is greater than a material strength of the core, and where a material strength of the coating is less than the material strengths of both the shell and core.

### Segmentation/Compartmentalization

In embodiments, a multilayer lattice structure made by the methods herein disclosed can be segmented/compartmentalized along at least a portion of a length of the continuously bioprinted fiber that forms the lattice structure. Details regarding the production of segmented/compartmentalized fiber structures is described in U.S. Provisional Patent Application No. 63/192,552, the disclosure of which is expressly incorporated by reference herein in its entirety.

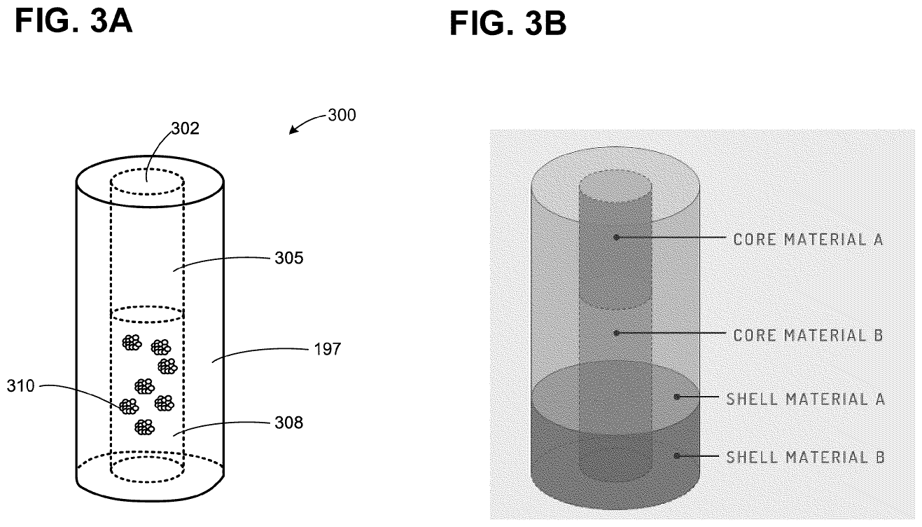

In embodiments, the core can be segmented/compartmentalized along at least a portion of the continuously bioprinted fiber that forms the lattice structure. In embodiments, at least one shell can be segmented/compartmentalized along at least a portion of the continuously bioprinted fiber that forms the lattice structure. In embodiments, both the core and the at least one shell may be segmented/compartmentalized along at least a portion of the continuously bioprinted fiber that forms lattice structure. In embodiments, one or more segments/compartments of the core and/or shell(s) may comprise pancreatic islets). Turning to FIG. 3, depicted is an exemplary fiber 300 (e.g., continuous fiber) that can make up a lattice structure (e.g., lattice structure 100, lattice structure 150, lattice structure 165) of the present disclosure. In this example illustration, the solid core 302 comprises first compartment 305 and second compartment 308. Second compartment 308 comprises pancreatic islets 310, while first compartment 305 does not. While not explicitly illustrated, it may be understood that first compartment 305 may comprise a different material (e.g., different hydrogel material) than that of second compartment 310, although in other embodiments the material composition may be the same. Furthermore, a shell can additionally or alternatively be segmented/compartmentalized, in similar fashion (see representative illustration of FIG. 3B), depending on the desired application.

Compartment sizing may be a function of one or more variables, including but not limited to fiber size (e.g., length and/or diameter), number of shell(s) and/or coating(s), type of materials used in the process of continuous fiber generation, coating composition, and the like. In some embodiments, a fiber may be comprised of at least two segments/compartments which include pancreatic islets, where other segments flanking the at least two segments/compartments are free of pancreatic islets. For example and without limitation, at least two segments comprising pancreatic islets may be included in the core of a continuously bioprinted fiber of the present disclosure. In another example, at least two segments comprising pancreatic islets may be included in the at least one shell. In embodiments, segment(s) comprising pancreatic islets may be of greater, equal, or lesser length(s) than segment(s) lacking the cells. In some embodiments, spacing between compartments/segments inclusive of biological material (e.g., cells) in a fiber of the present disclosure may be between 1-5 mm, for example 1 mm, 2 mm, 3 mm, 4 mm or 5 mm apart.

Other design considerations for compartmentalization can, by example, include optimizing oxygen and nutrient diffusion for improved viability and function as well as switching core and/or shell of a fiber structure between different materials, cell types and densities. Compartmentalization can also permit printing of a fiber structure with a different therapeutic dose without changing the geometry of the fiber structure.

Segments/compartments may comprise biological materials, for example, pancreatic islets of particular densities. In embodiments, the density may be the same or different between compartments. In embodiments, the biological material in compartments may be the same, or may be different. In embodiments, density of the biological material may be selected as a function of one or more of particular application (e.g., treatment of particular disease/condition), cell viability determinants, material (e.g., biocompatible material) in which the biological material is included, and the like.

In embodiments, one or more segments/compartments comprising biological material may be flanked by segments that comprise, for example, materials with immunoprotective properties. For illustrative purposes and without limitation, an immunoprotective hydrogel material may comprise, for example, a functionalized alginate including but not limited to methacrylated alginate, alginate furan, alginate thiol, alginate maleimide, and covalent click alginates (e.g., alginate blended with DMAPS-Alg and/or DMAPS-Hzd). For example, in a case in which the core of a lattice structure as herein disclosed includes one or more segments comprising biological material, the one or more segments may be flanked by other segments that comprise immunoprotective materials as herein disclosed. In another example, in a case in which a shell of a lattice structure as herein disclosed includes one or more segments comprising biological material, the one or more segments may be flanked by other segments that comprise immunoprotective materials as herein disclosed. It is also within the scope of this disclosure that where a core of a bioprinted fiber comprises biological material, the at least one shell and/or at least one coating can comprise materials with immunoprotective properties. In another embodiment where a shell of a lattice structure comprises biological material, another shell and/or at least one coating can comprise materials with immunoprotective properties.

### Input Materials

Aspects of the invention include input materials that can be used for printing lattice structures for advantageous use as biomaterials. “Biomaterial” as used herein refers to a natural or synthetic substance that is useful for constructing or replacing tissue, e.g. human tissue with or without living cells. In the field of bioprinting, the term “biomaterial” is often synonymous with the term “bioink.” A number of such materials have been described above, with further elaboration below.

An input material will generally comprise at least one cross-linkable material, e.g., hydrogels including but not limited to, alginate (e.g., SLG-100 alginate), chitosan, PEGDA, PEGTA, Hyaluronic acid (HA), HAMA, collagen, CollMA, gelatin, gelMA, agarose, gellan, fibrin (fibrinogen), PVA, and the like, or any combination thereof, as well as non-hydrogels including but not limited to, PCL, PLGA, PLA, and the like, or any combination thereof. In preferred embodiments an input material comprises at least one hydrogel. Non-limiting examples of hydrogels include alginate, agarose, collagen, fibrinogen, gelatin, chitosan, hyaluronic acid-based gels, or any combination thereof. A variety of synthetic hydrogels are known and can be used in embodiments of the systems and methods provided herein. For example, in some embodiments, one or more hydrogels form at least part of the structural basis for three-dimensional structures that are printed. In some embodiments, a hydrogel has the capacity to support growth and/or proliferation of one or more cell types, which may be dispersed within the hydrogel or added to the hydrogel after it has been printed in a three dimensional configuration.

In embodiments, a hydrogel is cross-linkable by a chemical cross-linking agent. For example, a hydrogel comprising alginate may be cross-linkable in the presence of a divalent cation such as calcium chloride (CaCl₂), a hydrogel containing chitosan may be cross-linked using a polyvalent anion such as sodium tripolyphosphate (STP), a hydrogel comprising fibrinogen may be cross-linkable in the presence of an enzyme such as thrombin, and a hydrogel comprising collagen, gelatin, agarose or chitosan may be cross-linkable in the presence of heat or a basic solution.

In embodiments hydrogel fibers may be generated through a precipitation reaction achieved via solvent extraction from the input material upon exposure to a cross-linker material that is miscible with the input material. Non-limiting examples of input materials that form fibers via a precipitation reaction include collagen and polylactic acid (PLA). Non-limiting examples of cross-linking materials that enable precipitation-mediated hydrogel fiber formation include polyethylene glycol (PEG) and alginate. Cross-linking of the hydrogel will increase the hardness of the hydrogel, in some embodiments allowing formation of a solidified hydrogel.

In some embodiments, a hydrogel comprises alginate. Alginate forms solidified colloidal gels (high water content gels, or hydrogels) when contacted with divalent cations. Any suitable divalent cation can be used to form a solidified hydrogel with an input material that comprises alginate. In the alginate ion affinity series Cd²⁺>Ba²⁺>Cu²⁺>Ca²⁺>Ni²⁺>Co²⁺>Mn²⁺, Ca²⁺ is the best characterized and most used to form alginate gels (Ouwerx, C. et al., Polymer Gels and Networks, 1998, 6(5):393-408). Studies indicate that Ca-alginate gels form via a cooperative binding of Ca²⁺ ions by poly G blocks on adjacent polymer chains, the so-called “egg-box” model (ISP Alginates, Section 3: Algin-Manufacture and Structure, in Alginates: Products for Scientific Water Control, 2000, International Specialty Products: San Diego, pp. 4-7). G-rich alginates tend to form thermally stable, strong yet brittle Ca-gels, while M-rich alginates tend to form less thermally stable, weaker but more elastic gels. In some embodiments, a hydrogel comprises a depolymerized alginate.

In some embodiments, a hydrogel is cross-linkable using a free-radical polymerization reaction to generate covalent bonds between molecules. Free radicals can be generated by exposing a photoinitiator to light (often ultraviolet), or by exposing the hydrogel precursor to a chemical source of free radicals such as ammonium peroxodisulfate (APS) or potassium peroxodisulfate (KPS) in combination with N,N,N,N-Tetramethylethylenediamine (TEMED) as the initiator and catalyst respectively. Non-limiting examples of photo cross-linkable hydrogels include: methacrylated hydrogels, such as hyaluronic acid methacrylate (HAMA), gelatin methacrylate (GEL-MA) or polyethylene (glycol) acrylate-based (PEG-Acylate) hydrogels, which are used in cell biology due to their inertness to cells. Polyethylene glycol diacrylate (PEG-DA) is commonly used as scaffold in tissue engineering, since polymerization occurs rapidly at room temperature and requires low energy input, has high water content, is elastic, and can be customized to include a variety of biological molecules.

In embodiments, an input material comprises a non-biodegradable polymer. In examples the input material may be a synthetic polymer, for example polyvinyl acetate (PVA). In embodiments, an input material may comprise hyaluronic acid (HA).

In some embodiments, a hydrogel comprises a chemically modified alginate. In examples, the chemically modified alginate comprises alginate functionalized with methacrylate groups, referred to herein as “Alg-MA.” In some embodiments, the Alg-MA can be used in an immunoprotective shell layer via blending with zwitterionic alginate, referred to herein as “Alg-zw.” Because of a dual cross-linking capability of Alg-MA, in embodiments the Alg-MA may be first printed with Alg-zw via physical cross-linking. Upon printing the fibers can then be further irradiated to induce covalent cross-linking across fibers thus resulting in F-F adhesion. In some embodiments, the chemically modified alginate may comprise thiolated alginate.

In some embodiments, one or more synthetic components may be added into hydrogel materials. Synthetic components may be useful in increasing fiber-to-fiber adhesion and/or in vivo stability. In examples, a material may comprise an acrylated zwitterionic monomer (e.g., sulfobetaine methacrylate (SBMA) and a cross-linker (e.g., poly(ethylene glycol) diacrylate (PEGDA). In such an example, photomediated cross-linking of the zwitterionic monomer with PEGDA may render the resultant cross-linked polymer matrix superhydrophilic, and hence, less prone to foreign body response (see U.S. Provisional Patent Application No. 63/192,552, the contents of which is expressly incorporated by reference herein in its entirety).

In some embodiments, hydrogel materials may be cross-linked via click chemistry. For example, copolymers comprising a zwitterionic monomer and aldehyde motifs (e.g., [2-(Methacryloyloxy)ethyl]dimethyl-(3-sulfopropyl)ammonium hydroxide (DMAPS)-aldehyde, referred to herein as “DMAPS-Ald”), and zwitterionic monomer and hydrazide motifs (e.g., DMAPS-hydrazide, referred to herein as “DMAPS-Hzd”), may be used (see U.S. Provisional Patent Application No. 63/192,552). Aldehyde reacts readily with hydrazide, forming covalently cross-linked hydrogels. Because of the presence of zwitterionic monomer in the polymer backbone, these polymers may exhibit low protein binding properties. In embodiments, one of these polymers may be blended with alginate in a shell. Following printing, the structure may be submersed in a solution containing the counter component that will in turn result in a covalently cross-linked bridge between fibers leading to F-F adhesion.

In embodiments, an input material comprises microparticles, “Microparticles” as used herein refers to immiscible particles in the range of about 0.1 um to about 100 um that are typically composed of a polymer, a metal, or other inorganic material. They can be symmetrical (e.g. spherical, cubic, etc) although this is not a requirement. Microparticles having an aspect ratio of 2:1 or greater may be considered a microrod or microfibre.

Input materials in accordance with embodiments herein can comprise any of a wide variety of natural or synthetic polymers that support the viability of living cells, including, e.g., alginate, laminin, fibrin, hyaluronic acid, poly(ethylene) glycol based gels, gelatin, chitosan, agarose, or combinations thereof. In some embodiments, the subject bioink compositions are physiologically compatible, i.e., conducive to cell growth, differentiation, communication, and other various cell functions (e.g., release of insulin). In certain embodiments, an input material comprises one or more physiological matrix materials, or a combination thereof. By “physiological matrix material” is meant a biological material found in a native mammalian tissue. Non-limiting examples of such physiological matrix materials include: fibronectin, thrombospondin, glycosaminoglycans (GAG) (e.g., hyaluronic acid, heparin sulfate, chondroitin-6-sulfate, dermatan sulfate, chondroitin-4-sulfate, or keratin sulfate), deoxyribonucleic acid (DNA), adhesion glycoproteins, and collagen (e.g., collagen I, collagen II, collagen III, collagen IV, collagen V, collagen VI, or collagen XVIII). Such physiological matrix material may contribute to, e.g., pancreatic β-cell survival, proliferation, and/or insulin secretion in the context of lattice structures of the present disclosure (Riopel M, and Wang R., Frontiers in Bioscience (Landmark Ed). (2014); 19(1): 77-90); Nikolova G et al., Dev Cell. (2006); 10(3): 397-405; Johansson A et al., Diabetologia. (2009); 52(11): 2385-94).

### Additional Fluids

Aspects of the invention include one or more buffer solutions. Buffer solutions in accordance with embodiments of the invention are miscible with an input material (e.g., a hydrogel) and do not cross-link the input material. In some embodiments, a buffer solution comprises an aqueous solvent. Non-limiting examples of buffer solutions include polyvinyl alcohol, water, glycerol, propylene glycol, sucrose, gelatin, or any combination thereof.

Buffer solutions in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a buffer solution can be modulated so that it matches the viscosity of one or more input materials.

Aspects of the invention include one or more sheath fluids. Sheath fluids in accordance with embodiments of the invention are fluids that can be used, at least in part, to envelope or “sheath” an input material being dispensed from a dispensing channel. In some embodiments, a sheath fluid comprises an aqueous solvent. Non-limiting examples of sheath fluids include polyvinyl alcohol, water, glycerol, propylene glycol, sucrose, gelatin, or any combination thereof. Sheath fluids in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a sheath fluid can be modulated so that it matches the viscosity of one or more input materials.

In some embodiments, a sheath fluid comprises a chemical cross-linking agent. In some embodiments, a chemical cross-linking agent comprises a divalent cation. Non-limiting examples of divalent cations include Cd²⁺, Ba²⁺, Cu²⁺, Ca²⁺, Ni²⁺, Co²⁺, or Mn²⁺. In a preferred embodiment, Ca²⁺ is used as the divalent cation. In some embodiments, the concentration of a divalent cation in the sheath fluid ranges from about 80 mM to about 140 mM, such as about 90, 100, 110, 120 or 130 mM.

### Cell Populations

In embodiments, the cell population is selected from the group comprising or consisting of a single-cell suspension, cell aggregates, cell spheroids, cell organoids, or combinations thereof. Input materials in accordance with embodiments of the invention can incorporate any mammalian cell type, including but not limited to stem cells (e.g., embryonic stem cells, adult stem cells, induced pluripotent stem cells), germ cells, endoderm cells (e.g., lung, liver, pancreas, gastrointestinal tract, or urogenital tract cells), mesoderm cells (e.g., kidney, bone, muscle, endothelial, or heart cells), ectoderm cells (skin, nervous system, pituitary, or eye cells), stem cell-derived cells, or any combination thereof. In preferred embodiments, at least one cell population comprises or consists of insulin-producing cells, for example pancreatic islets comprising or consisting of β-cells (e.g., insulin-producing) and, in embodiments one or more of α-cells (e.g., glucagon-producing), δ-cells (e.g., somatostatin-producing), PP-cells (e.g., pancreatic polypeptide-producing) and ε-cells (e.g., ghrelin-producing).

Cells can be obtained from donors (allogenic), from a different species to the recipient (xenogeneic), or from recipients (autologous). Specifically, in embodiments, cells can be obtained from a suitable donor, such as a human or animal, or from the subject into which the cells are to be implanted. Mammalian species include, but are not limited to, humans, monkeys, dogs, cows, horses, pigs, sheep, goats, cats, mice, rabbits, and rats. In one embodiment, the cells are human cells. In other embodiments, the cells can be derived from animals such as dogs, cats, horses, monkeys, or any other mammal.

In some embodiments, pancreatic islets for inclusion in lattice structures of the present disclosure comprise re-aggregated islets. Re-aggregated islets can be formed from the dispersing of native-islets, or in some examples stem cell derived β-cells, into single cell suspensions, followed by re-aggregation of the dispersed cells into uniform (optionally smaller) “pseudoislets.” Techniques for producing pseudoislets are known (see e.g., Lu, Jia et al., JURA. (2016-2017); 6: 1-4). Creating aggregates of uniform size is important for reproducible production of functional insulin-producing cells. Several groups have developed methods to reproducibly create endocrine cell aggregates of defined size to improve functionality (Gao B et al., Acta Mech. Sin. (2019); 35: 329-337; Nair G et al., Nat. Cell Biol. (2019); 21: 263-274; Velazco-Crus L et al., Stem Cell Rep. (2019); 12: 351-365). The production of uniformly sized cell aggregates in a scalable manner can enable the production of mature cells for cell therapies such as herein disclosed, in a consistent manner. In some embodiments, cells may deposit insoluble ECM proteins and, in examples, contribute to or dynamically change surrounding microenvironment.

Because insulin-producing cells within the lattice structures herein disclosed are protected from the host immune system, pancreatic islets can be derived from any suitable source, i.e., human or non-human. In embodiments, the islet cells are stem or progenitor cells, including induced pluripotent stem cells that differentiate into insulin producing islet cells. Suitable insulin secreting cell populations and methods for producing such populations are known in the art, see e.g., U.S. Pat. Nos. 8,425,928; 5,773,255; 5,712,159; 6,642,003; Rezania et al., Nat. Biotech. (2014); 32: 1121-1133; and Kuo et al., Int'l. J. Clin. Med. (2014); I(I):21-25, each of which are hereby incorporated by reference in their entirety.

In some embodiments, the at least one biological material included in a lattice structure of the present disclosure comprises a cell population expressing/secreting one or more endogenous biologically active agent(s), e.g., insulin, glucagon, ghrelin, pancreatic polypeptide, Factor VII, Factor VIII, Factor IX, alpha-1-antitrypsin, an angiogenic factor, a growth factor, a hormone, an antibody, an enzyme, a protein, an exosome, and the like. Discussed herein, endogenous biologically active agents comprise those agents that the cell naturally produces in a biological context (e.g., insulin release in response to elevated glucose concentrations). An endogenous biologically active agent can constitute a therapeutic agent in the context of the present disclosure.

In some embodiments, an input material can comprise genetically engineered cells that secrete specific factors. It is within the scope of this disclosure that a cell population as discussed above can comprise, in embodiments, engineered cells (e.g., genetically engineered cells) that secrete specific factors. Cells can also be from established cell culture lines, or can be cells that have undergone genetic engineering and/or manipulation to achieve a desired genotype or phenotype. In some embodiments, pieces of tissue can also be used, which may provide a number of different cell types within the same structure.

Genetic engineering techniques applicable to the present disclosure can include but are not limited to recombinant DNA (rDNA) technology (Stryjewska et al., Pharmacologial Reports. 2013; 65: 1075), cell-engineering based on use of targeted nucleases (e.g., meganuclease, zinc finger nucleases (ZFN), transcription activator-like effector nucleases (TALEN), clustered regularly interspaced short palindromic repeat-associated nuclease Cas₉ (CRISPR-Cas₉), etc. (Lim et al., Nature Communications. 2020; 11: 4043; Stoddard B L, Structure. 2011; 19(1): 7-15; Gaj et al., Trends Biotechnol. 2013; 31(7): 397-405; Hsu et al., Cell. 2014; 157(6): 1262; Miller et al., Nat Biotechnol. 2010; 29(2): 143-148), cell-engineering based on use of site-specific recombination using recombinase systems (e.g., Cre-Lox) (Osborn et al., Mol Ther. 2013; 21(6): 1151-1159; Hockemeyer et al., Nat Biotechnol. 2009; 27(9): 851-857; Uhde-Stone et al., RNA. 2014; 20(6): 948-955; Ho et al., Nucleic Acids Res. 2015; 43(3): e17; Sengupta et al., Journal of Biological Engineering. 2017; 11(45): 1-9), and the like. In some embodiments, some combination of the above-mentioned techniques for cell-engineering may be used.

Encompassed by the present disclosure are engineered cells capable of producing one or more therapeutic agents, including but not limited to proteins, peptides, nucleic acids (e.g., DNA, RNA, mRNA, siRNA, miRNA, nucleic acid analogs), peptide nucleic acids, aptamers, antibodies or fragments or portions thereof, antigens or epitopes, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, small molecules, drugs (e.g., drugs, dyes, amino acids, vitamins, antioxidants) or any combination thereof.

In embodiments, cells of the present disclosure may be modified to comprise at least one mechanism for providing a local immunosuppression at a transplant site when transplanted in an allogeneic host, for example in tissue fibers of the present disclosure. In examples, a cell or cells may comprise a set of transgenes, each transgene encoding a gene product that is cytoplasmic, membrane bound, or local acting, and whose function can include but is not limited to mitigate antigen presenting cell activation and function; to mitigate graft attacking leukocyte activity or cytolytic function; to mitigate macrophage cytolytic function and phagocytosis of allograft cells; to induce apoptosis in graft attacking leukocytes; to mitigate local inflammatory proteins; and to protect against leukocyte-mediated apoptosis (WO2018/227286; Harding et al., BioRxiv. 2019; DOI: 10.1101/716571; Lanza et al., Nature Reviews Immunology. 2019; 19: 723-7331; Harding et al., Cell Stem Cell. 2020; 27(2): 198-199).

In embodiments, cells of the present disclosure may be modified in a manner to exert control over cell proliferation. As an example, a cell may be genetically modified at a cell division locus (CDL) to comprise a negative selectable marker and/or an inducible activator-based gene expression system, thereby enabling control over the permitting, ablation and/or inhibition of proliferation of the genetically modified cells by addition or removal of an appropriate inducer (WO2016/141480; Liang et al., Nature. 2018; 563(7733): 701-704).

Appropriate growth conditions for mammalian cells are well known in the art (Freshney, R. I. (2000) Culture of Animal Cells, a Manual of Basic Technique. Hoboken N.J., John Wiley & Sons; Lanza et al. Principles of Tissue Engineering, Academic Press; 2nd edition May 15, 2000; and Lanza & Atala, Methods of Tissue Engineering Academic Press; 1st edition October 2001). Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS) or calf serum and 100 U/ml penicillin are appropriate as are various other standard media well known to those in the art. Growth conditions will vary depending on the type of mammalian cells in use and the tissue desired.

In some embodiments, cell-type specific reagents can be advantageously employed in the subject input materials for use with a corresponding cell type. For example, an ECM can be extracted directly from a tissue of interest and then solubilized and incorporated it into an input material to generate tissue-specific input materials for printed tissues. Such ECMs can be readily obtained from patient samples and/or are available commercially from suppliers such as zPredicta (rBone™, available at zpredicta.com/home/products).

### Printing Systems

In preferred embodiments, bioprinting systems comprise technology as described in WO2014/197999, WO2018/165761, WO2020/056517, WO2021/081672, and U.S. Provisional Patent Application No. 63/290,595, the disclosures of which are expressly incorporated herein by reference. As detailed therein, the disclosed bioprinting systems and components thereof enable multi-material switching, and hence the composition of one or more components (e.g., cell type, biomaterial composition) of the synthetically generated tissue fiber can be modified along the length of the fiber while continuously printing. In embodiments, a microfluidics-based bioprinting system is the RX1™ bioprinter (Aspect Biosystems, Vancouver, BC, Canada), or the system described in co-pending U.S. Provisional Patent Application No. 63/290,595, the contents of which is incorporated by reference herein in its entirety.

In an exemplary embodiment of a preferred bioprinting system, the system comprises a print head comprising a dispensing channel, wherein one or more material channels and a core channel converge at the proximal end of the dispensing channel. A print head may be configured to dispense buffer solution and/or sheath fluid simultaneous with one or more cross-linkable materials. In some embodiments, a print head is configured to maintain a constant mass flow rate through the dispensing channel. In this manner, a print head can be configured to facilitate a smooth and continuous flow of one or more input materials (or a mixture of one or more input materials) and a buffer solution and/or sheath fluid through the dispensing channel. In use of such a print head, an input material flowing through the dispensing channel can be cross-linked from the inside, by a fluid flowing through the core channel and/or from the outside, by sheath fluid flowing through a downstream sheath fluid channel, as described more particularly in WO2020/056517. In some embodiments, a print head comprises one or more fluidic focusing chambers comprised of a conical frustum shape and, optionally, one or more print head adaptors, as described in detail in WO 2021/081672, and U.S. Provisional Patent Application No. 63/290,595. In embodiments, a print head is the DUO™ microfluidic printhead, or the CENTRA™ microfluidic printhead (Aspect Biosystems, Vancouver, BC, Canada).

Other examples of bioprinting systems relevant in the context of the present disclosure, for example those that can be modified or used in conjunction with the methods of the present disclosure, include but are not limited to 3-D Bioplotter® (EnvisionTEC Inc., Dearborn, MI, USA), NovoGen Bioprinter® Platform (Organovo®, San Diego, CA, USA), R-Gen 100 and R-Gen 200 (RegenHU, Villas-Saint-Pierre, Switzerland), Bioprinter Fabion and Fabion 2 (3D Bioprinting Solutions, Moscow, Russia), BioBot® Basic, BioAssemblyBot® 200/400/500 (Advanced Solutions, Louisville, KY, USA), BIO X™, BIO X6™, INKREDIBLE+™ (CellINK, Boston, MA, USA), Ourobotics Revolution (Ourobotics, Cork, Ireland), BioScaffolder 2.1 (GeSim, Radeberg, Germany), Omega Bioprinter (3Dynamic Systems, Bridgend, UK), Syn\hat{ } and Explorer (Bio3D, Singapore), Alevi 1/2/3 (Alevi by 3D Systems, Rock Hill, SC, USA), and Dr. Invivo 4D6 (Rokit Healthcare, Seoul, South Korea).

### Methods of Making

Aspects of the invention include methods of printing a three-dimensional (3D) device comprising two or more layers of planar structures having a desired infill density. The general manner of printing a first layer and, optionally a second layer, third layer, and so on, is described in detail above with reference to FIGS. 1A-3B.

Turning to FIG. 4, depicted is an exemplary process flow for fabricating a multilayer lattice structure of the present disclosure. Broadly speaking, the process flow includes generating a lattice structure from a continuous fiber, preferably via a fabrication platform (e.g., frame 210 at FIG. 4), and then applying at least one coating to obtain the implantable device. In the exemplary process flow of FIG. 4, the continuous fiber is produced in a manner whereby the continuous fiber is exposed to a sheath fluid containing cross-linker during printing that cross-links the continuous fiber from the outside in, but in other embodiments inside-out cross-linking where cross-linker is included as part of a core material is within the scope of this disclosure (see U.S. Provisional Patent Application No. 63/192,552).

Step (1) at FIG. 4 comprises printing the continuous fiber. In this example, during printing the continuous fiber includes a core 402 comprising pancreatic islets 408, and shell 404 surrounding said core, which in turn is surrounded by sheath fluid 406 comprising a cross-linker. In embodiments, the shell 404, and optionally the core 402, comprises a cross-linkable material. Although depicted as one shell, fibers comprising more than one shell are within the scope of this disclosure. As the fiber is printed, the sheath fluid is removed, for example via flowing through a porous receiving surface (not shown). Following bioprinting of the lattice structure, step (2) optionally comprises submerging the lattice 410 in cross-linking solution 412 (or otherwise applying cross-linking solution, for example via spraying or dispensing via the same dispensing means used to dispense the continuous fiber) to facilitate/continue uniform cross-linking of the entire printed lattice structure.

Step (3) is divided into two sub-steps (3a) and (3b). Step (3a) includes coating the entire lattice structure 410 with coating solution 416. The coating at step (3a) can be applied, for example, by submerging lattice structure 410 into a coating solution, by using a microfluidic print head (e.g., the same microfluidic print head used to fabricate the fiber) to dispense coating solution 416 onto the completed lattice structure 410, spraying the lattice structure 410 with coating solution 416, and the like. In embodiments, the coating solution 416 comprises a cross-linkable material (e.g., alginate). In embodiments, the coating solution 416 comprises a material that is the same as the material comprising the core 402 and/or shell 404 of the lattice structure 410 being coated. In other embodiments, the coating solution 416 may comprise a material that is different than the material comprising the core 402 and/or shell 404. In embodiments, at step (3a), residual cross-linker (e.g., Ca²⁺) associated with the lattice structure 410 contributes to initial cross-linking of the material in the coating solution 416 with the material comprising the shell 404. Following coating of the lattice structure 410, step (3b) includes submerging the entire conformal coated lattice structure 418 in cross-linking solution 412 (or otherwise applying cross-linking solution, for example via spraying or dispensing via the same microfluidic print head used to dispense the continuous fiber). While the illustrated embodiment at FIG. 4 includes use of the same cross-linking solution 412 to cross-link the lattice 410 and coated lattice structure 418, it is within the scope of this disclosure that different cross-linking solutions can be used, for example in a case where the material used to fabricate a shell (e.g., shell 404) is comprised of a different material than the material used to fabricate a coating (e.g., coating solution 416). In this way, via the process flow depicted at FIG. 4, a coating 420 is added uniformly to an entirety of a lattice produced from a continuously bioprinted fiber, to produce the coated lattice structure 418 as shown. Although not specifically illustrated, it is within the scope of this disclosure that a second coating can be further applied in similar fashion as that of the first coating.

In some embodiments where a single coating is applied to a lattice, the coating may have impart stability and/or F-F adhesion properties and/or anti-FBR properties to the resultant fiber structure. In some embodiments where a fiber structure comprises two coatings, a first coating may have particular desired properties (e.g., materials selected for enhancing stability and/or F-F adhesion) while the second coating may have additional/alternative properties (e.g., tailored to have anti-FBR properties. In preferred embodiments, the coating is a conformal coating that uniformly covers the entirety of the exposed surfaces of the fiber structure.

In embodiments, a coating (e.g., 420) is comprised of a hydrogel material, for example a hydrogel material comprising one or more of alginate, zwitterionic alginate, SBMA, chitosan, PEGDA, PCL, PEG, poly-L-lysine (PLL), PEGTA, Hyaluronic acid (HA), HAMA, collagen, CollMA, gelatin, gelMA, agarose, gellan, fibrin (fibrinogen), PVA, and the like, or any combination thereof. In some examples, the coating is comprised of a functionalized alginate, i.e., an alginate that is chemically modified to include one or more properties, including but not limited to immunoprotective properties that are advantageous in the manufacture of lattice structures of the present disclosure. Examples of functionalized alginates include but are not limited to methacrylated alginate, alginate furan, alginate thiol, alginate maleimide, and covalent click alginates (e.g., alginate blended with DMAPS-Alg and/or DMAPS-Hzd).

In embodiments, a coating (e.g., coating 420 at FIG. 4) is of a material strength that is less than the material strength of one or more shell(s) (e.g., shell 404 at FIG. 4) and/or a core (e.g., core 402 at FIG. 4). In some embodiments where a lattice structure comprises two coatings, an outer coating may be of a material strength that is lesser than a material strength of an inner coating, or vice versa. In some embodiments where a lattice structure comprises two coatings, an outer coating and an inner coating may be of a substantially same material strength, optionally where the outer coating and inner coating are comprised of different materials. In embodiments, a core (e.g., 402) is solid, optionally wherein the core is segmented/compartmentalized along the length of the fiber. In additional or alternative embodiments at least one shell may be segmented/compartmentalized along the length of the fiber. In embodiments, a lower material strength of a coating, preferably an outer coating (in the case of two or more coatings) contributes to reduced FBR response when the lattice structure is implanted into a subject.

The conformal coating may impart stability to the fiber structure, and/or may endow the structure with properties that optimize the interface between the fiber structure and host, e.g. anti-FBR properties, promotion of vascularization, etc. In embodiments, the coating may be softer than the outermost external shell. In embodiments, the core, external shell, and conformal coating may comprise from about 0.1% to about 4% alginate. In embodiments, the fiber structure may comprise a core of about 0.75%-1.5% alginate, an external shell of about 1.5-2.5% alginate, and a conformal coating of about 0.2-2.5%, such as 0.2-0.75%, alginate.

In embodiments wherein two coatings are applied, it is within the scope of this disclosure that the innermost coating may be of a greater hardness than outermost coating. In embodiments, the core, external shell, and conformal coatings may comprise from about 0.1% to about 4% alginate. In embodiments, the fiber structure may comprise a core of about 0.75%-1.5% alginate, an external shell of about 1.5-2.5% alginate, an inner conformal coating of about 1.5-2.5% alginate, and an outer conformal coating of about 0.2-2.5%, such as 0.2-0.75%, alginate. Such examples are meant to be illustrative. Further details on the production of such conformal coatings is described in U.S. Provisional Patent Application No. 63/342,118, the contents of which is expressly incorporated by reference herein in its entirety.

In embodiments, a bioprinted fiber structure made by the methods herein disclosed can also be segmented/compartmentalized along at least a portion of a length of a fiber, preferably a continuous fiber, that makes up the bioprinted fiber structure. Details of the production of segmented/compartmentalized bioprinted fiber structures is described in U.S. Provisional Patent Application No. 63/192,552, the contents of which is expressly incorporated by reference herein in its entirety.

In some embodiments, a bioprinted lattice structure as herein disclosed comprises a solid core comprising about 1.5% alginate, a shell comprising about 0.25% to about 3.0% alginate, and a conformal coat comprising about 0.25% alginate to about 1.5% alginate. In a preferred embodiment, a bioprinted lattice structure comprises a solid core comprising about 1.5% alginate, a shell comprising about 2% alginate, and a coating comprising about 0.5% alginate.

In some embodiments, a method first comprises providing a design for a lattice structure as herein disclosed to be printed. The design can be created using commercially available CAD software. In some embodiments, the design comprises information regarding specific materials (e.g., for heterogeneous structures comprising multiple materials) to be assigned to specific locations in the structure(s) to be printed.

In embodiments, a method comprises dispensing a continuous fiber as herein disclosed into a lattice, and then coating the lattice to produce the desired lattice structure. In some embodiments, dispensing the continuous fiber comprising dispensing the continuous fiber via the use of a fabrication platform as described in co-pending Provisional Application No. 63/342,118. In embodiments, a method comprises providing a bioprinting system comprising a fabrication platform for supporting a lattice structure during printing, patterning, or processing, the fabrication platform comprising a frame (210 at FIG. 2) comprising a plurality of posts for securing a continuous fiber during printing; at least one dispensing orifice for dispensing the continuous fiber; a positioning unit for positioning the fabrication platform in three dimensional space with respect to the at least one dispensing orifice; and at least one dispensing means for dispensing the continuous fiber from the at least one dispensing orifice. The method may comprise, via the bioprinting system, dispensing the continuous fiber around a plurality of said posts to form a lattice of at least two, three, four, or five layers of the continuous fiber, followed by coating the resulting lattice structure with at least one coating. In some embodiments, the bioprinting system comprises at least one print head comprising a plurality of microfluidic printing channels to selectively provide a respective plurality of materials.

### Methods of Treatment

The bioprinted 3D lattice structures of the present disclosure are useful in methods of treating diabetes in a subject in need thereof. In one embodiment, a method of treating diabetes treats a subject with Type 1 diabetes (i.e., insulin-dependent diabetes). In an embodiment, the method of treating diabetes treats a subject with any one of Type 1, MODY, LADA, brittle, lean, Type 1.5, Type 2, Type 3, obesity related diabetes, or any combination thereof. In embodiments, the subject is a human subject, preferably wherein the human subject is suffering from Type 1 diabetes.

The methods involve providing a bioprinted lattice structure as herein described, said lattice structure comprising a therapeutically effective amount of pancreatic islets (e.g., re-aggregated pancreatic islets) encapsulated within said lattice structure. The therapeutically effective amount of pancreatic islets may comprise some predetermined therapeutically effective number of insulin-producing cells (i.e., β-cells). In embodiments, the therapeutically effective amount of pancreatic islets comprises between about 20,000 and 80,000 IEQs, more preferably between about 25,000 and 75,000 IEQs, most preferably about 30,000, about 40,000, about 50,000 or about 60,000 IEQs. In embodiments, therapeutically effective amounts of pancreatic islets corresponding to a fiber device can vary according to, for example, the size and health of the individual being treated. For example, a therapeutically effective amount of pancreatic islets corresponding to a fiber device can comprise some number/amount of islets, or IEQs per kilogram of body weight.

The methods involve implanting a lattice structure as herein disclosed into a subject in need thereof. In embodiments the methods comprise implanting a lattice structure into a subject one or more times. For example, in embodiments the methods comprise implanting into a subject in need thereof a lattice structure comprising a therapeutically effective amount of pancreatic islets about once every 4 months, once every 6 months, once a year, once every two years, once every three years, once every four years, once every five years, or more. In embodiments, the pancreatic islets included in the lattice structure survive at least for at least four months, or at least 6 months, or at least one year, or at least two years, or at least three ye The conformal coating may impart stability to the fiber structure, and/or may endow the structure with properties that optimize the interface between the fiber structure and host, e.g. anti-FBR properties, promotion of vascularization, etc. In embodiments, the coating may be softer than the outermost external shell. In embodiments, the core, external shell, and conformal coating may comprise from about 0.1% to about 4% alginate. In embodiments, the fiber structure may comprise a core of about 0.75%-1.5% alginate, an external shell of about 1.5-2.5% alginate, and a conformal coating of about 0.2-2.5%, such as 0.2-0.75%, alginate.

In embodiments wherein two coatings are applied, it is within the scope of this disclosure that the innermost coating may be of a greater hardness than outermost coating. In embodiments, the core, external shell, and conformal coatings may comprise from about 0.1% to about 4% alginate. In embodiments, the fiber structure may comprise a core of about 0.75%-1.5% alginate, an external shell of about 1.5-2.5% alginate, an inner conformal coating of about 1.5-2.5% alginate, and an outer conformal coating of about 0.2-2.5%, such as 0.2-0.75%, alginate. Such examples are meant to be illustrative.

In embodiments, a bioprinted fiber structure made by the methods herein disclosed can be segmented/compartmentalized along at least a portion of a length of a fiber, preferably a continuous fiber, that makes up the bioprinted fiber structure. Details of the production of segmented/compartmentalized bioprinted fiber structures is described in U.S. Provisional Patent Application No. 63/192,552, the contents of which is expressly incorporated by reference herein in its entirety. ars, or at least four years, or at least five years, or more, following implantation. In some embodiments, a subject may require just a single implant. In some embodiments, the lattice structure may need to be replaced once every 6, 7, 8, 9, 10, 11, 12, 13, 14, 15, 16, 17, or 18 months, once every 1, 2, 3, 4, 5 or more years, or until the subject has recurring hyperglycemia, or a return to the diabetic state.

In some embodiments, the lattice structure comprising the therapeutically effective amount of pancreatic islets are implanted to the greater omentum of the subject. The greater omentum (also known as the great omentum, omentum majus, gastrocolic omentum, epiploon, or, caul) is a large apron-like fold of visceral peritoneum that hangs down from the stomach and extends from the greater curvature of the stomach back to ascend to the transverse colon before reaching to the posterior abdominal wall. Thus, the lattice structure may be implanted into a pouch formed surgically from the omentum.

In one embodiment, the lattice structure can be surgically implanted using minimally invasive surgical techniques such as laparoscopy. In embodiments, the lattice structure is implanted laparoscopically into the abdominal cavity or thoracic cavity. In some embodiments, the implanting is carried out intraperitoneally, percutaneously, or subcutaneously.

In some embodiments, the lattice structure is anchored or immobilized (e.g., by suture) at the implantation site to maintain the lattice structure near the implantation site. In embodiments, the delivery of the therapeutic agent (e.g., insulin) is not location dependent and biodistribution of the agent is dependent on the subject's vasculature or body fluids. In embodiments, the lattice structure is implanted percutaneously or subcutaneously under the skin of the abdomen, forearm, flank, back, buttocks, leg, and the like, where it substantially remains until such time as it is required to be removed.

In embodiments, the lattice structure is retrievable after implantation. In some embodiments, anchoring or immobilizing the lattice structure prevents the lattice structure from migrating, moving, or traversing inside the subject, and facilitates easy retrieval. However, it is also within the scope of this disclosure that given the dimensions of the lattice structure, anchoring or otherwise immobilizing the lattice structure may not be needed, where retrieval remains easily facilitated. Retrieval may be desirable after the pancreatic islets held within the lattice structure cease or substantially cease to release the therapeutic agent (e.g., insulin), after the release of the therapeutic agent drops below some predetermined threshold (e.g., lower than some acceptable amount), after cell death exceeds or is expected to exceed some cell death threshold, and the like.

Following retrieval, the retrieved fiber device can be replaced by another fiber device to maintain the desired effect, for example the release of insulin from pancreatic islets housed therein, in response to elevated levels of blood glucose.

Particular treatment regimens comprising implanting the disclosed lattice structures may be evaluated according to whether they will improve a given patient's outcome, meaning it will help stabilize or normalize the subject's blood glucose levels or reduce the risk or occurrence of symptoms or co-morbidities associated with diabetes, including but not limited to, episodes of hypoglycemia, elevated levels of glycosylated hemoglobin (HbA1C levels), heart disease, retinopathy, neuropathy, renal disease, hepatic disease, periodontal disease, and non-healing ulcers.

Thus, for the purposes of this disclosure, a subject is treated if one or more beneficial or desired results, including desirable clinical results, are obtained or are expected to be obtained. For example, beneficial or desired clinical results include, but are not limited to, one or more of the following: decreasing one or more symptoms resulting from diabetes, increasing the quality of life of those suffering from diabetes, decreasing the dose of other medications required to treat diabetes, delaying or preventing complications associated with diabetes, and/or prolonging survival of individuals.

Furthermore, while the subject of the methods is generally a subject with diabetes, the age of the patient is not limited. The disclosed methods are useful for treating diabetes across all age groups and cohorts. Thus, in some embodiments, the subject may be a pediatric subject, while in other embodiments, the subject may be an adult subject.

In embodiments, use of the lattice structure by the diabetic patient will substantially decrease the need to monitor blood sugar levels and may eliminate the need for insulin injections altogether. It is within the scope of this disclosure that the implanted lattice structure may be monitored regularly (e.g., weekly, bi-monthly, monthly) to ensure the cells of the lattice structure are functioning as desired. For example, in embodiments the lattice structure can comprise one or more contrast agents to facilitate in vivo monitoring of fiber device placement, location of implant at some time-point after implantation, health of the implant, deleterious effects of non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and other known to those of skill in the art.

Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implanted lattice structure.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Bioprinted Cell Therapy Platform Normalizes Blood Glucose Control in Diabetic Rat

We have developed microfluidic bioprinting technology that combines biocompatible materials with clinically relevant cells to manufacture implantable tissues for therapeutic applications. Islet cell therapy has been clinically validated for type 1 diabetes (T1D) but relies on lifelong immune-suppression and is limited by the supply of cadaveric donor islets. We are developing a bioprinted pancreatic tissue therapeutic that can deliver allogeneic islets or stem cell-derived pancreatic beta cells into T1D patients without the need for immune-suppression, by encapsulation in materials that support physiologic function and shield these cells from direct host immune cell attack.

This Example demonstrates bioprocessing methods to package primary islets into bioprinted tissue implants for in vitro testing and in vivo function studies. We evaluated the ability of bioprinted human islet tissues (xenogeneic) to restore blood glucose control in diabetic, immune-deficient mice and adapted this process for a bioprinted primary rat islet tissue (allogeneic) delivered to the omentum of diabetic rats. Finally, we developed a process to scale this bioprinted pancreatic tissue manufacturing process for delivery of implants in large animals and humans.

### Materials and Methods

The main procedure that was done on the animals is omental device implantation as outlined below. Surgeries were performed post-treatment with STZ.

### A. Prepping the Animal Into Surgery.

General concepts of “Rodent Anesthesia” (SOP ACC-01-2017), “Analgesia for Adult Mice and Rats Meloxicam SOP” (TECH 19), and “Local Anesthesia/Analgesia in Adult Mice and Rats Bupivacaine SOP” (TECH 16), were followed. The procedure is explained below:
A1. Place animal in induction chamber on heating pad (temperature should be approximately 38° C.) and induce anesthesia with isoflurane. Flush chamber and move animal to maintenance circuit on nose cone and heat support and maintain isoflurane anesthesia.
A2. Administer a small drop of lubricating eye gel into each eye. Lay the animal flat on its stomach and administer supportive care fluids in the form of 0.9% saline or Lactated Ringers Solution (LRS) at 20 mL/kg subcutaneously. use a 25 G with various syringe sizes depending on the dose.
A3. Administer 1 mg/kg Metacam subcutaneously.
A4. Administer buprenorphine 0.05 mg/kg subcutaneously.
A5. While flipping animal over place paper towels under it to catch all the shaved hair. Shave abdominal skin of the animal with a hair clipper.
A6. Take a dry piece of gauze and remove all the hair off and around animal while pulling the paper towel out at the same time.
A7. Once the hair is cleaned up, take a piece of gauze or cotton swab soaked in soap to clean the shaved area. The gauze needs to be moist but not dripping and it will be wrapped around finger. Clean the shaved area in a circular motion and outward, the bubbles should be visible. Leave the soap on the animal for about 30 seconds before wiping down with alcohol.
A8. Wipe the shaved area with gauze or cotton swab in 70% alcohol in an outward circular motion. Go in with a new gauze or cotton swab dipped in soap again but this time scrub/rub the oil on the skin off. Let it sit for 30 second before proceeding to the next step.
A9. Clean the area off with a new gauze or cotton swab soaked in alcohol.
A10. To inject local anesthetic as a line block at the planned incision site. Lift the skin and insert needle subcutaneously under the skin. Pull the needle out as you inject with a “bleb” forming.
A110. Perform one additional skin prep (gauze dipped in soap and again with alcohol). Toe pinch the animal to ensure it is properly anesthetized. Change gloves and wash hands with soap.
A12. Throughout procedure, monitor colour of extremities (should be pink), breathing rate and depth and toe pinch every 5 minutes.

### B. Prepping Surgical Instruments

B1. Don clean exam gloves. This surgery is performed using sterile tip technique, the gloved hands cannot come into contact with any surface that must remain sterile.
B2. Open sterile pack on the counter and aseptically remove the sterile field/wrap that is folded in half, open it up so the inside (sterile field) is placed facing up when placing it close to the surgery table.
B3. Take a pair of sterile forceps to transfer all the instruments and supplies in the pack onto the sterile field wrap. Only the sterile part of the instruments can go on to the sterile field.
B4. Take “Glad Press and Seal Wrap”, throw the first 6″ out, and pull out a piece to place on top of the animal. Avoid using the edges as they are contaminated. Make sure not to touch the top surface with fingers.
B5. Use a pair of forceps to pick up cotton pad to help stick down the Press and Seal onto the animal. To pick up Press and Seal, use a sterile 25 G needle to puncture close to the surgery incision area and grab it with forceps to cut a hole over the planned incision site. Do not allow anything non-sterile to come into contact with the top surface of the drape.
B6. Toe pinch the animal with forceps to ensure it is at a surgical level of anesthesia.

### C. Surgical Procedures.

General concepts of “Rodent Survival Surgery” (SOP ACC-02-2017) were be followed. All surgical procedures on immune-deficient rats were performed inside a biosafety cabinet of a laminar flow clean air workstation. The procedure is explained below:
C1. Use a pair of toothed forceps to pick up skin. Make a 20 mm incision along the midline of the skin a ˜2 cm below the xiphoid process using a scalpel blade.
C2. Once the skin incision is complete, grasp and lift muscle with the forceps and first make a stab opening using scalpel blade and then make a 20 mm incision with the scissors.
C3. Use a self-retaining tissue retractor to keep the incision to the peritoneal cavity open.
C4. Aseptically place a sterile gauze on the abdominal skin caudal to the opening to prevent direct contact of omental membrane with skin.
C5. Locate the greater omentum and gently stretch the omental membrane out of the opening using a pair of tissue forceps.
C6. Aseptically place the bioprinted implant in the middle of the exposed omentum.
   Note: bioprinted implants are composed of a non-reactive, non-rigid polymer with no known bio-incompatibilities. Each device is 20×20×2.5 mm and may contain cells, but is specifically designed to prevent cell release. One device is implanted in each animal. The implants are printed using sterile medical grade ingredients. (The cells are incorporated during the printing process.) Following printing, the devices are be maintained in standard cell culture conditions (culture medium, 37° C./5% CO2) for no more than four days before implantation. Just prior to implantation, the device is washed with a sterile isotonic solution such as saline or Ringer's buffer to remove all traces of the medium.
C7. Fold free ends the omental membrane over the implant and sutured two sides of the omentum using a fine non-absorbable monofilament (e.g., 6-0 Nylon or Prolene), to create a closed pouch.
C8. Gently slide back the omental pouch into the abdominal cavity, and the incision closed with continuous or subcuticular sutures for the muscle and skin layers respectively.
C9. Wet the 5-0 absorbable suture with saline. Use 1 suture pack per rat. Hold the needle perpendicularly with a pair of needle drivers. Make sure the suture does not touch the non-sterile area of the needle holder or forceps.
C10. Insert the needle into one side of the muscle, take it out from the other side of the muscle. When taking the needle out, go with the direction of the needle. Tie a square knot and repeat this knot a total of 3 times. Leave about 2-3 mm of suture and cut the ends. Repeat until muscle areas are closed.
C11. Perform a subcuticular closure of the skin. On one side of the skin, insert the needle just under the skin layer and take it out on a farther side (deeper but still under skin layer) of the skin. Insert the needle on the opposite side of the skin, go from deep to superficial this time. Ensure the needle does not come out of on top of the skin. Tie square knot three times.
C12. Once the skin layer is closed, take a 25 G needle and dip into Gluture. Take the needle covered with Gluture to place on top of the sutured skin. Use a pair of forceps to pinch the skin around the needle and slowly take the needle out. The sutured area is now properly closed.

### Recovery of the Rat:

C13. Turn off isoflurane and leave the rat on oxygen via nose cone.
C14. Gently remove the drape and clean any blood off the rat.
C15. Once rat has recovered its righting reflex, place rat into the warmed recovery cage (paper towel covering the bare bottom of the cage).
C16. Monitor the rat in the recovery cage until fully recovered from anesthesia and able to maintain its body temperature without supplemental heat (i.e.: eating, drinking, walking normally, able to climb on a hut, able to groom).
C17. Once fully recovered, return the rat to normal housing cage with cage mates.

### D. Analgesia Plan

D1. Pre-operative Analgesics: Rats will receive a local anesthetic (bupivacaine 0.5 mg, 200 ul of 2.5 mg/ml solution) at the site of incision before tissue is cut, and NSAID (Meloxicam 1 mg/kg, SC), and buprenorphine (0.05 mg/kg, SC) injections. Rats with liver disease induction will receive a combination of oral Ibuprofen and low dose Buprenorphine to account for reduced liver metabolism in these liver disease animals. Oral Ibuprofen (30 mg/kg; resuspension of Ibuprofen liquigel capsule in water by vortexing, protecting from light, replaced every 3 days) will be administered at the time of surgery according to TECH 09b Oral dosing (Gavage) in Rats.
D2. Post-operative Analgesics: Day 1 and Day 2: Meloxicam (1 mg/kg, SC, SID) and buprenorphine (0.02 mg/kg, SC, BID)
   TECH 16 (Local Anesthesia/Analgesia in Adult Mice and Rats Bupivacaine SOP) and TECH 19 (Analgesia for Adult Mice and Rats Meloxicam SOP) were followed for these procedures. Rats with liver disease induction will receive a combination of oral Ibuprofen and low dose Buprenorphine to account for reduced liver metabolism in these liver disease animals. Oral Ibuprofen (30 mg/kg; resuspension of Ibuprofen liquigel capsule in water by vortexing, protecting from light, replaced every 3 days) will be administered 6 hours post-surgery according to TECH 09b Oral dosing (Gavage) in Rats. Overnight, the animals continued to receive Ibuprofen in their drinking water (1 mg/mL). At 24 and 48 hours post-surgery, the animals received a subcutaneous injection of Buprenorphine (0.05 mg/kg).

### E. Prophylactic Antibiotic Administration

The bioprinted implants were prepared using sterile materials and reagents and in a sterile condition. All surgical procedures were also performed in an aseptic manner. However, to mitigate any potential risk of infection, prophylactic antibiotics (Enrofloxacin 100 μg/mL in drinking water) was added to drinking water of rats 3 days before surgery until 3 days after surgery. Enrofloxacin is mainly excreted by the kidneys and is also suitable for rats with liver disease.

### Other Procedures

### F. Blood Sampling

Blood samples were collected from rats on a weekly basis until the endpoint of the study. Blood was collected form lateral saphenous vein using TECH 02 SOP “Blood collection from the lateral saphenous vein in mice and rats”. Maximum weekly blood sample volume was 150 ul which is within the allowed limit for sequential blood sampling defined in “UBC ANIMAL CARE COMMITTEE POLICY 006, Policy on Acceptable Methods of Rodent Blood Withdrawal”

### G. Streptozotocin Administration for Induction of Diabetes

Streptozotocin (STZ) destroys the insulin-secreting cells of the pancreas, thereby inducing diabetes. As use of STZ warrants special handling precautions, all users and animal care technicians were made aware of the hazards through the presence of an SDS in the experimental area. Cages containing animals treated with STZ were marked as such for 1 week following STZ injection.

STZ, which is supplied as a powder, is reconstituted in acetate or citrate buffer (pH 4.5) to a concentration of 30 mg/mL just prior to injection.
G1. To induce a model of type 1 diabetes (insulin insufficiency), STZ was injected intraperitoneally rats at a dose of 60 mg/kg in acetate or citrate buffer (a maximum volume of ˜0.5 mL for a standard 250 g rat). TECH 10b (Intraperitoneal Injection in the Adult Rat) was followed for IP injection procedure.
G2. Following injection, the animals' blood glucose levels was monitored daily. Those animals in which sustained hyperglycemia develops (>20 mmol/l blood glucose for two consecutive readings) were used for experiments.
   There is a risk of severe hypoglycemia in the first 24-48 hours. Initial cytotoxic destruction of the beta islet cells causes an excessive release of insulin into the bloodstream. To prevent any fatal hypoglycemia, sucrose water was provided during the induction period to reduce morbidity and mortality. To do so, 10% sucrose was added to drinking water for 48 hours post-STZ injection.

### H. Blood Glucose Measurements

Blood glucose were measured by placing a drop of blood (≤10 μL) onto a glucometer test strip (Lifescan Canada or equivalent).
H1. A small drop of blood was taken from tail vein using TECH 13 (UBC ACC Tail Poke SOP—Rat) and applied on the test strip.
H2. Apply gentle pressure with a 2×2 gauze to tip of tail for approximately 10 seconds to stop blood flow.

### I. Oral Glucose Tolerance Test

The main function of pancreatic islets is to secret insulin in response to increase blood sugar levels. To monitor in vivo function of implanted pancreatic islet, we stimulated islets with an oral glucose dose after a short fasting period as described below:
I1. Fast animal for 4 hours (e.g. from 8 AM to 12 PM).
I2. Take a blood sample (fasting, volume: 75 ul)
I3. Dose animal with glucose solution (3 g/kg, 150 ul) through oral gavage using using a 1 mL syringe and a flexible 20 gauge and 38 mm length feeding tube (TECH 09—Oral dosing in Mice and Rats will be followed)
I4. Take another blood sample after 30 min (post-glucose dosing, volume: 75 ul).

### J. Immunofluorescence Protocol for Insulin and CD31

### Materials:

Insulin (C27C9) Rabbit mAb (New England Biolabs), CD31 (PECAM-1) Mouse mAb (New England Biolabs), Anti-rabbit IgG, Alexa Fluor® 647 conjugate (New England Biolabs), Anti-mouse IgG, Alexa Fluor® 488 conjugate (New England Biolabs).

### Procedure:

1. Place slides in a 60° C. oven for 15 min to start deparaffinization process
2. Place slides in a xylene-resistant holder
3. Wash in xylene for 5 min (3×)
4. Wash in 100% EtOH for 5 min (2×)
5. Wash in 95% EtOH for 5 min
6. Wash in 80% EtOH for 5 min
7. Wash in 70% EtOH for 5 min
8. Wash in PBS, on a shaker for 5 min
9. Place slides in a beaker and cover with Antigen Retrieval Buffer (10 mM Citrate Buffer, pH 6.0)
10. Heat on HIGH power for 5 min (2×) in a microwave, making sure the slides are always covered in buffer
11. With heatproof gloves, remove beaker from the microwave and place in sink
12. Cool beaker for 5-10 min under a thin stream of cold running tap water (do not let water hit the slides directly)
13. Wash slides in ddH₂O for 5 min
14. Wash slides in PBS, on a shaker for 5 min
15. Remove excess water on slides and circumscribe samples with hydrophobic pen (Super Pap pen)
16. Block slides 1 hr at room temperature in 5% BSA-PBS with 5% goat serum in humidity chamber
17. Incubate with primary antibodies overnight at 4° C. in humid chamber (diluted in 5% BSA-PBS with 5% goat serum):
   a. Insulin: 1/500 dilution
   b. CD31: 1/500 concentration

### Day 3:

1. Wash slides in PBS for 10 min (3×)
2. Incubate with secondary antibodies at room temperature for 1 hr in dark humid chamber (diluted in 5% BSA-PBS or PBS):
   a. Anti-Mouse: 1/1000 dilution
   b. Anti-Rabbit: 1/1000 dilution
3. From this step forward, slides must be protected from light
4. Wash slides in PBS for 10 min (3×)
5. Mount slides with Fluoroshield with DAPI and seal coverslips with clear nail polish
6. Let dry 24 hours before imaging.

### Islet Processing & In Vitro Bioprinting

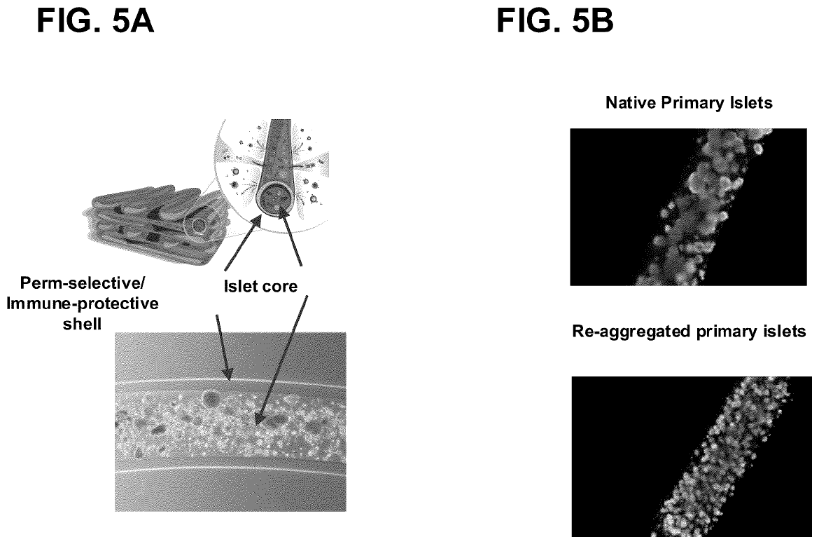

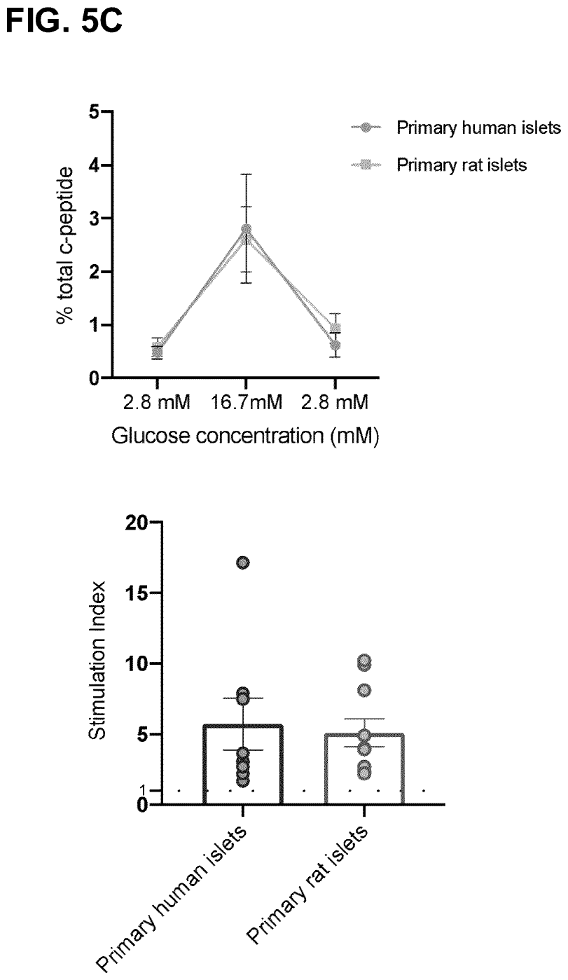

Depicted at FIG. 5A is a schematic representation & bright field image of bioprinted primary human islet tissue. FIG. 5B illustrates live/dead staining of bioprinted primary islets (top: native islets; bottom: re-aggregated islets) after 7 days in culture. FIG. 5C depicts data from a Glucose-Stimulated Insulin Secretion (GSIS) assay performed using primary human (n=8) and rat (n=10) islets; mean+/−SEM.

### Bioprinted Pancreatic Tissue Function in Streptozotocin-Induced Rodent Models of Diabetes

### Immune-Deficient (NSG) Mice: IP Implant

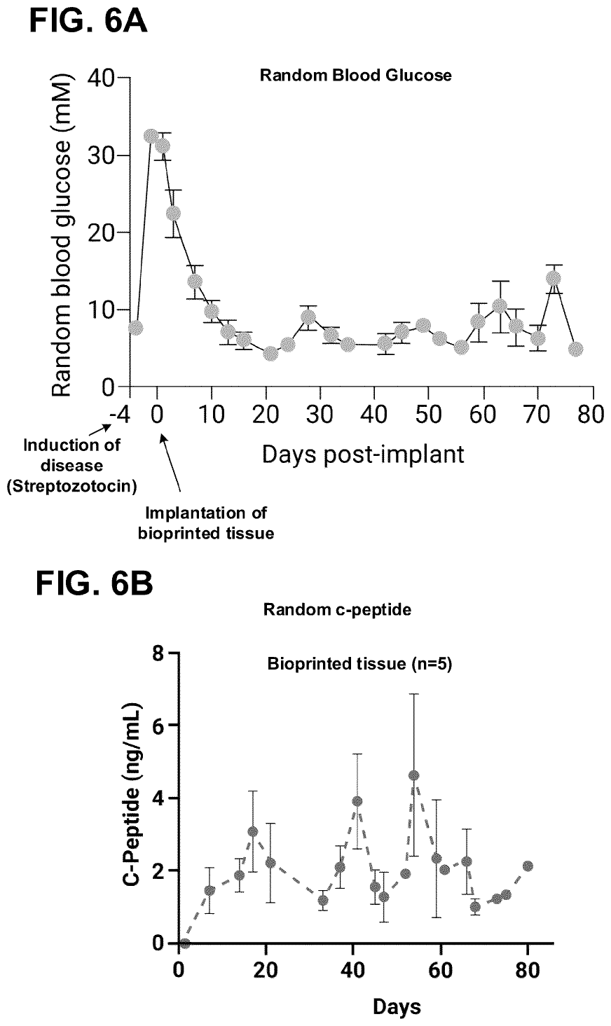

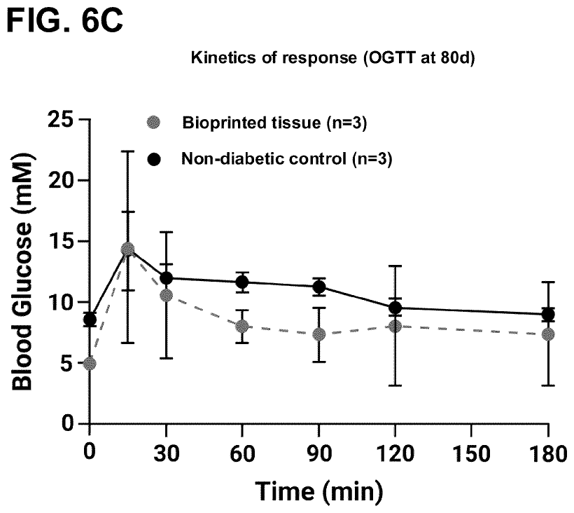

FIG. 6A illustrates random-fed blood glucose measurements following streptozotocin (STZ) treatment and intraperitoneal (IP) implantation of bioprinted human islet tissue in NSG (NOD scid gamma) mice (n=5) over 80 days; Day 0 represents time of implantation. FIG. 6B shows human C-peptide levels measured in mouse plasma over 80 days using ELISA. FIG. 6C illustrates data from an oral glucose tolerance test (OGTT) performed at day 80 to assess kinetics of blood glucose normalization following a fasting period and subsequent glucose challenge in NSG mice with bioprinted islet tissues or healthy, non-STZ treated control mice.

### Immune-Deficient (Nude) Rats: Omental Implant

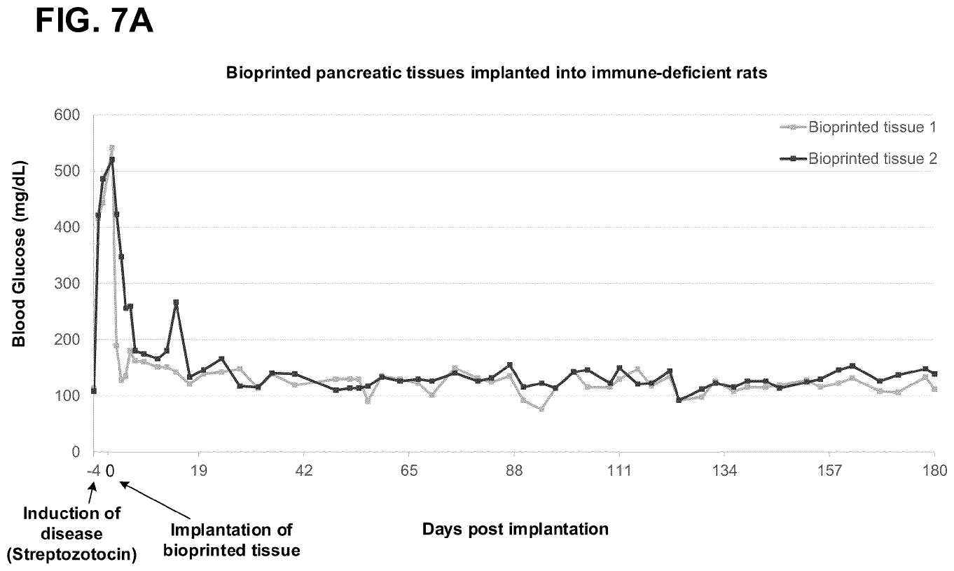

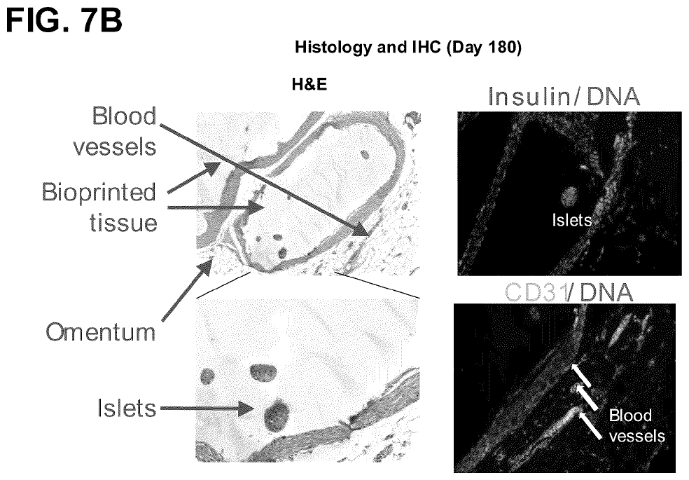

FIG. 7A illustrates blood glucose measurements following omental pouch implantation of bioprinted rat islet tissue in STZ-treated nude rats (n=2) over 180 days. FIG. 7B shows H&E (high and low magnification) and insulin (islets) or CD31 (endothelial cells) immunohistochemistry (IHC) performed on sections from fixed, bioprinted tissue explanted at 180 days.

### Immune-Competent (Sprague-Dawley) Rats: Omental Implant

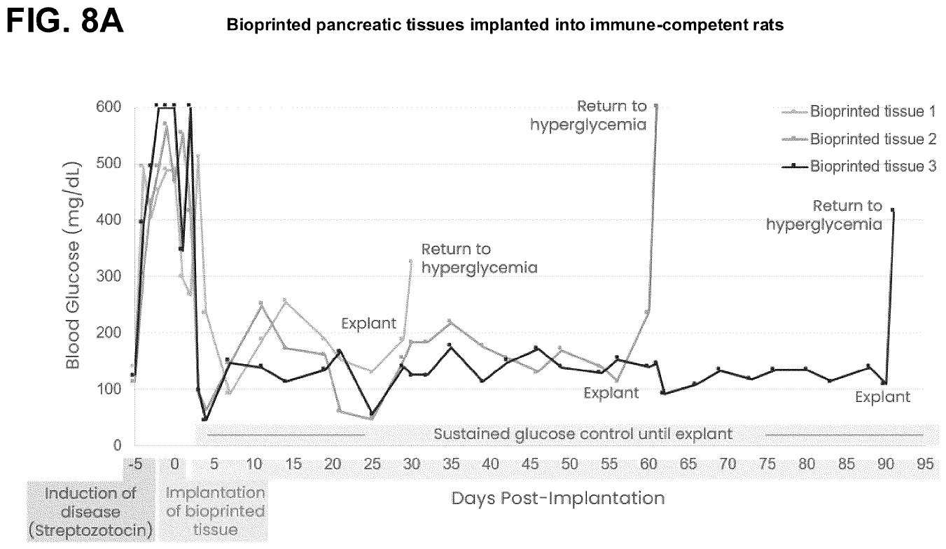

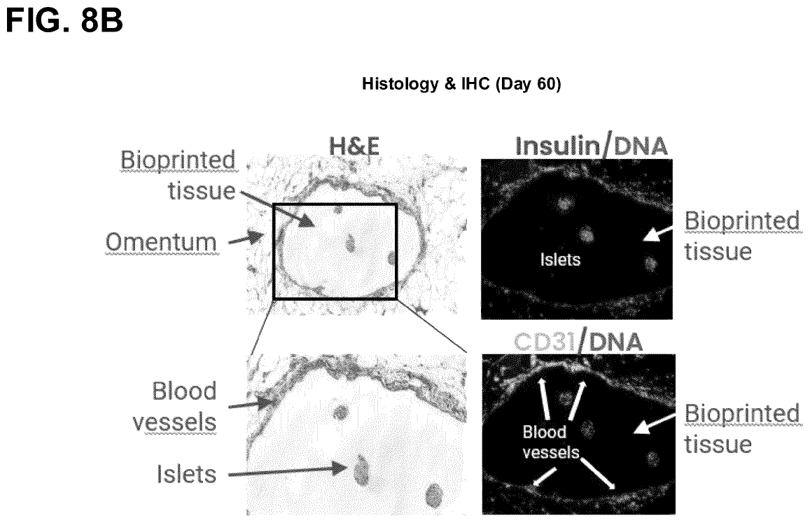

FIG. 8A illustrates blood glucose measurements following omental pouch implantation of bioprinted Lewis rat islet tissue in STZ-treated Sprague-Dawley (SD) rats (n=3) over 90 days; explant retrieval and return to hyperglycemia was performed at 30, 60, and 90 days following surgical implantation. FIG. 8B shows H&E and insulin (islets) or CD31 (endothelial cells) IHC performed on sections from fixed, bioprinted tissue explanted at 60 days.

### Bioprinted Pancreatic Tissue Scale-Up for Large Animals

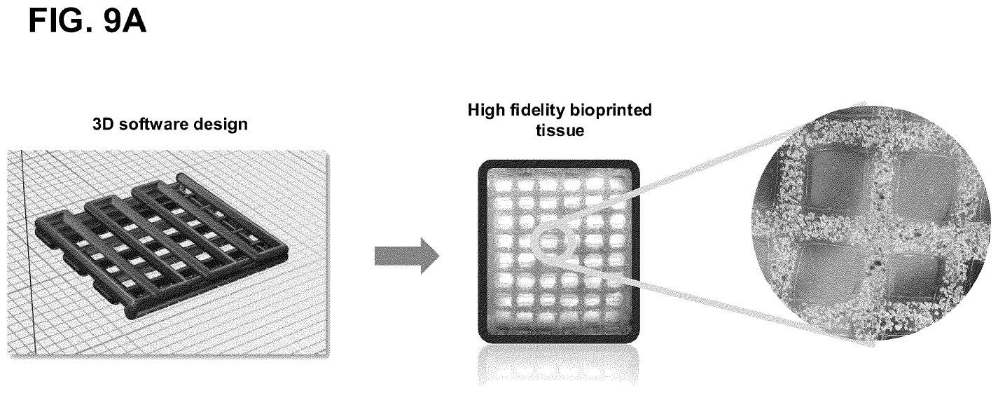

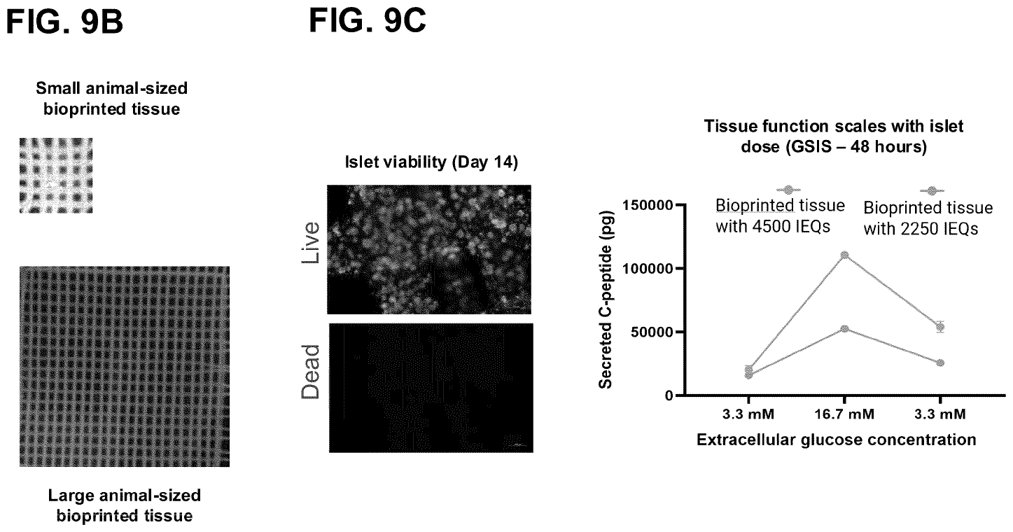

FIG. 9A is a schematic illustrating that the biomanufacturing process involves tissue design in proprietary software and QC of bioprinted tissues, including confirmation of micro- and macro-architectures, cell viability, and islet distribution. FIG. 9B shows bioprinted pancreatic tissue used for studies in rats compared to scaled-up tissue for large animals. FIG. 9C shows viability of bioprinted neonatal porcine islets confirmed up to 14 days post-print. GSIS demonstrates bioprinted tissue function scales with the dose of human islets.

### Conclusion

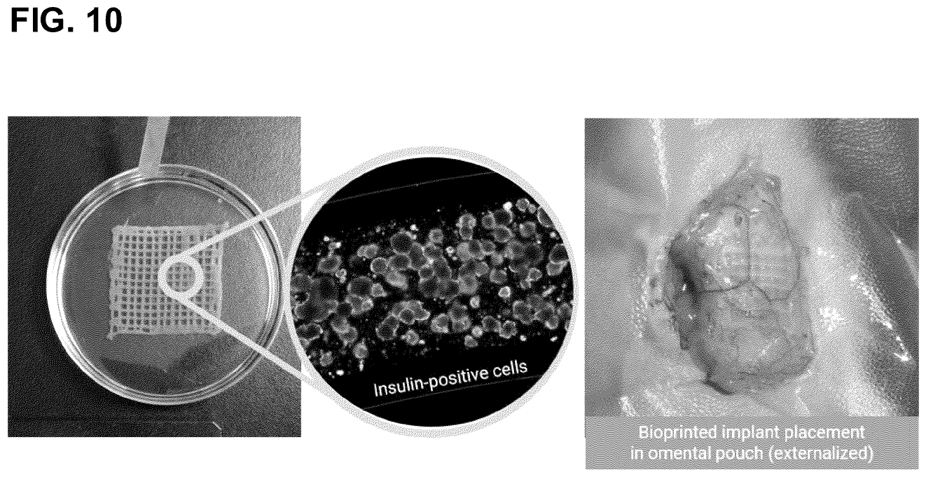

We developed a process (see FIG. 10) to manufacture implantable tissues containing bioprocessed pancreatic islets in materials that protect these allogeneic cells from host immune cell attack. This Example shows that the bioprinted pancreatic tissues: 1) maintain islet viability and function in vitro, 2) restore blood glucose control in mouse and rat models of diabetes, 3) support islet function and immune-protection over 90 days in a rat model of diabetes, and 4) can be scaled up for large animal studies and ultimately, delivery into T1D patients.

### Example 2. Printing Optimization and Viability Study of Mini Device

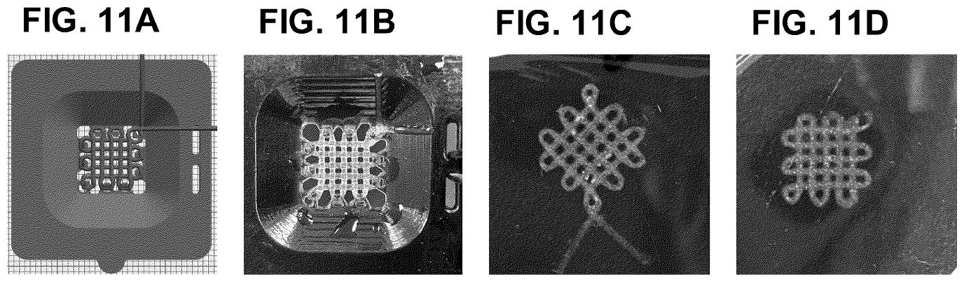

This Example included a test condition of 10×10 mm 4 layer lattice structure against a control condition of 18×18 mm 2 layer lattice structure. FIG. 11A is a schematic of the 10×10 mm lattice structure. FIG. 11B is an image of a coated 10×10 mm lattice structure coupled to a frame. FIGS. 11C-11D depict the coated 10×10 mm lattice structure uncoupled from the frame.

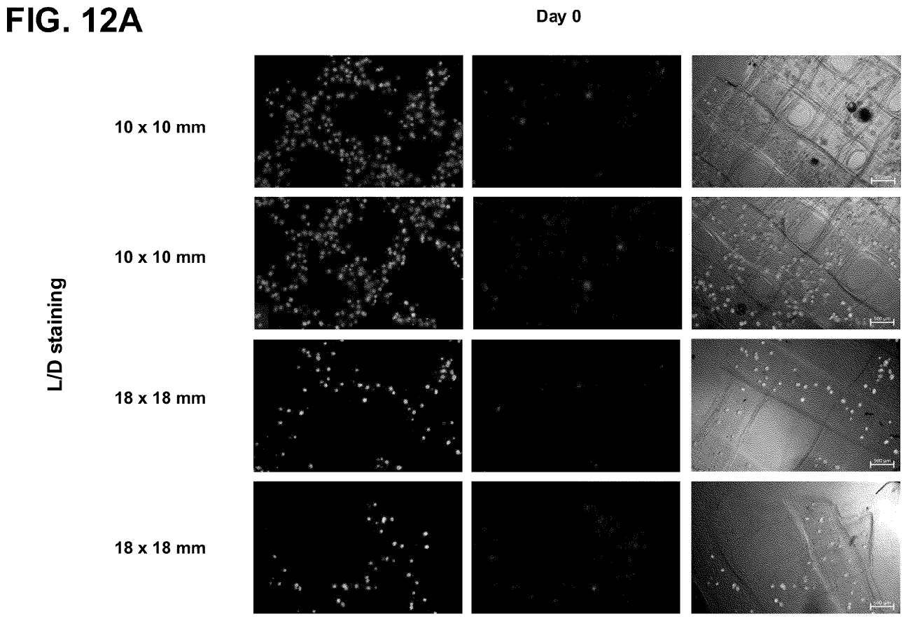

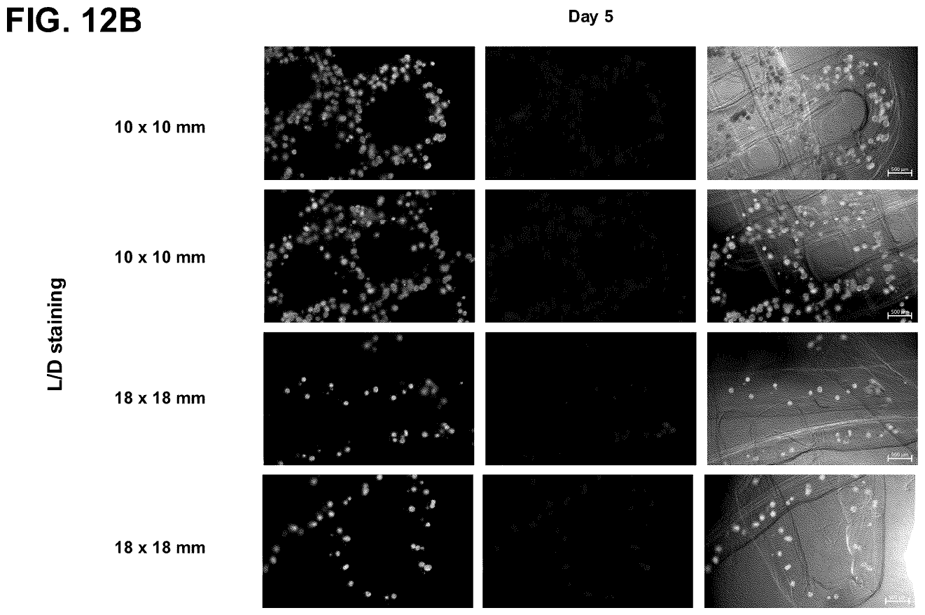

The devices were coated with 0.5% SLG100 (alginate). Cell dose for the devices was 3K IEQ HepG2 aggregates. Live/Dead staining was assessed 0 (FIG. 12A) and 5 days (FIG. 12B) following printing.

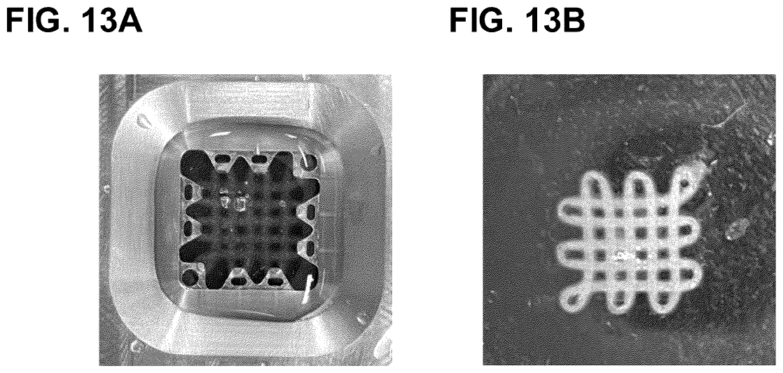

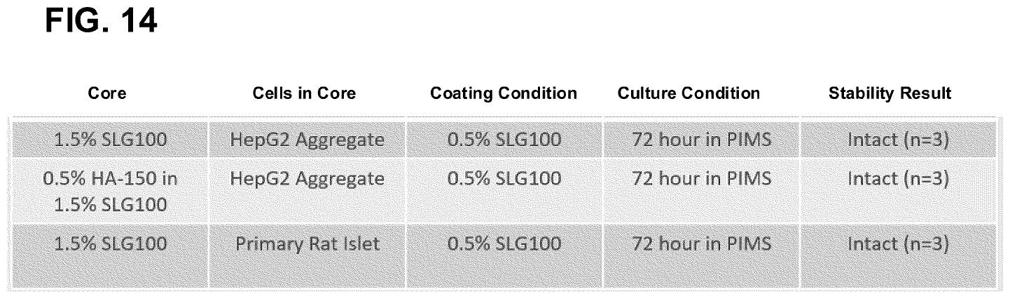

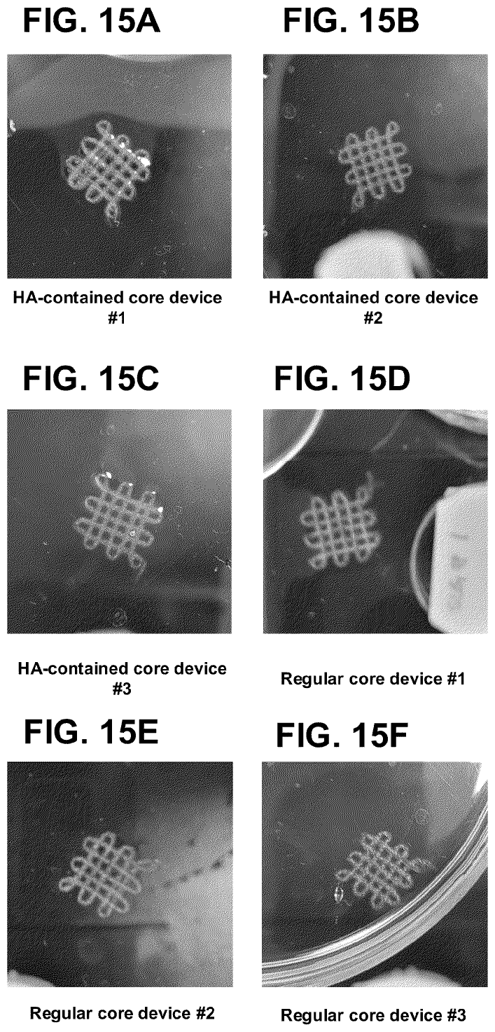

This Example also included testing of the mini device (10×10 mm, 4 layers device) with a coating of 0.5% SLG100, and a core of either HA-contained 1.5% SLG100 or just 1.5% SLG100. For each case, cell dose was 3K IEQ HepG2 aggregates or primary rat islets (PRI). FIGS. 13A-13B show images of the 10×10 mm lattice structure after coating, on frame (FIG. 13A) and uncoupled from frame (FIG. 13B). The stability data is summarized at FIG. 14. FIG. 15A is an image of structure 1 (HA-contained core), FIG. 15B is an image of structure 2 (HA-contained core), FIG. 15C is an image of structure 3 (HA-contained core), FIG. 15D is an image of structure 1 (regular core), FIG. 15E is an image of structure 2 (regular core), and FIG. 15F is an image of structure 3 (regular core).

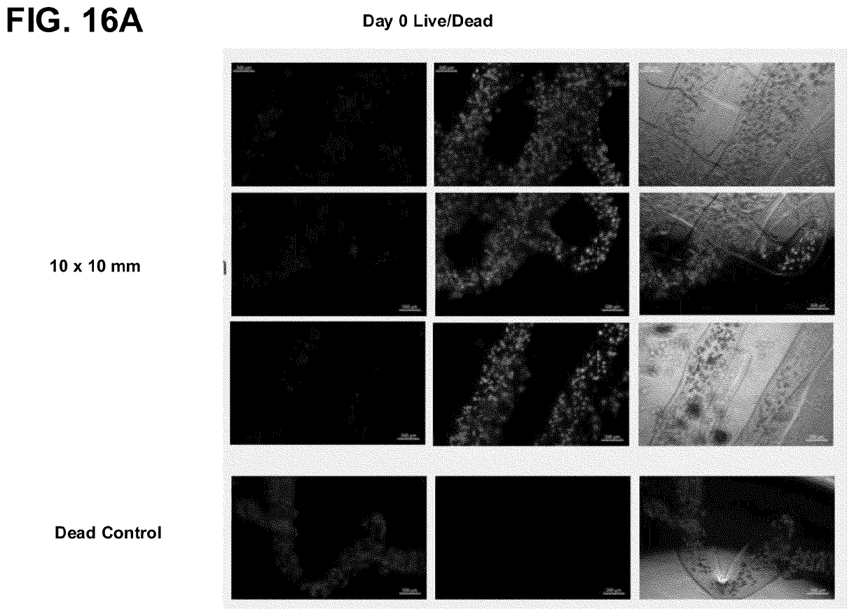

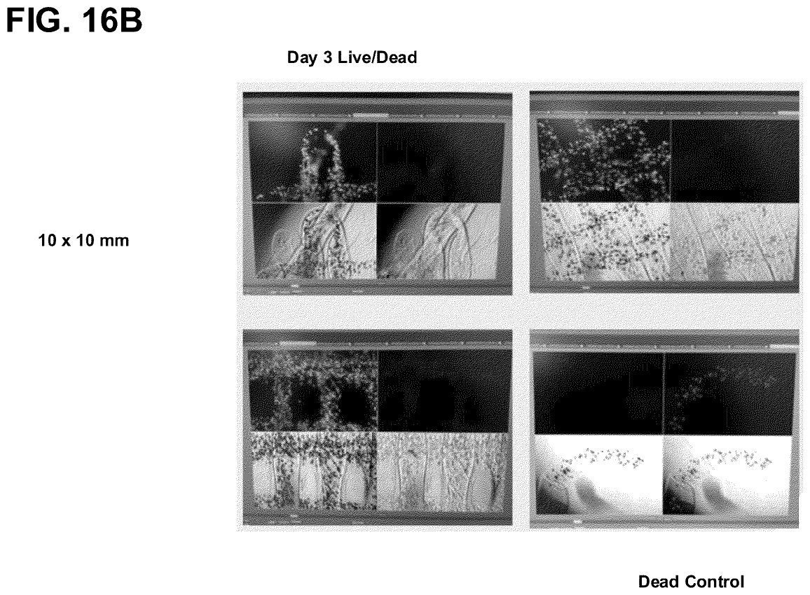

This Example also included testing of coated 10×10 mm devices loaded with PRI (coating 0.5% SLG100, cell dose: 3K IEQ PRI) for viability and functionality. Live/dead staining was assessed at 0 (FIG. 16A) and 3 (FIG. 16B) days post printing.

### Example 3. Frame vs Mesh Devices Stability Test

This Example demonstrates enhanced stability of a device printed via the use of a frame as compared to a device printed in lieu of a frame (i.e., onto mesh without use of a frame).

### Experimental Design

In this Example, the coated lattice structures tested were 18×18 mm, and 2 layers thick. The coated lattice structures included a core (1.5% SLG100), a shell (2% SLG100) and a conformal coating (0.5% SLG100). The core was printed at a flow rate of 115 μL/min, the shell was printed at 80 μL/min, and the sheath flow (i.e., cross-linker solution) was 55 μL/min as dispensed from the print head. Three structures were printed via the use of a frame as herein disclosed, whereas three other structures were printed onto mesh in lieu of a frame. The conformal coating was added to the bioprinted lattice structure while coupled to the frame (devices that relied on the frame for printing), or was added while the bioprinted lattice structure rested on the mesh (devices that were printed in lieu of a frame).

### Stability Test

The stability test was performed as follows. Each coated lattice structure was cultured in a 50 mL conical tube with 15 mL media for 3 days in PIMS media. Each coated lattice structure was subjected to an orbital shaking test at 125 rpm for 30 mins or car transportation for 30 mins. Next each coated lattice structure was poured out into a petri dish, and washed with 10 mL saline three times (saline was aspirated between each rinse). A spatula was used to lift and transfer each device between two petri dishes filled with saline to mimic device transferring on a surgical site. This was repeated 5 times. Finally, each coated lattice structure was transferred onto a moist plastic wrap, and a wand was used to move each device from one edge of the wrap to another side three times (to mimic repositioning of the device on omental membrane).

### Results

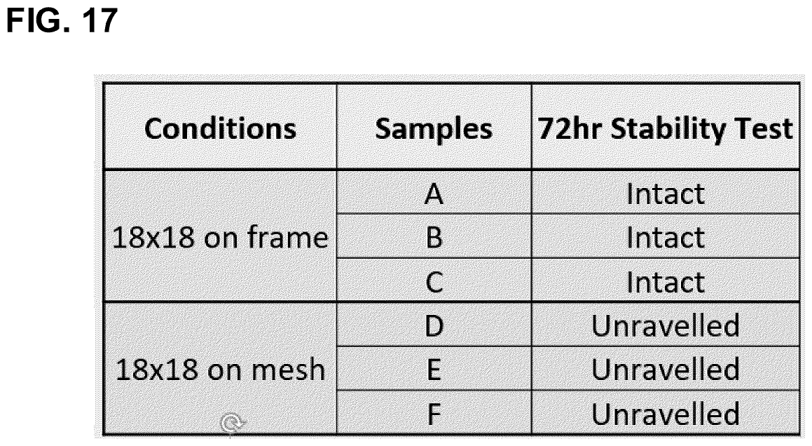

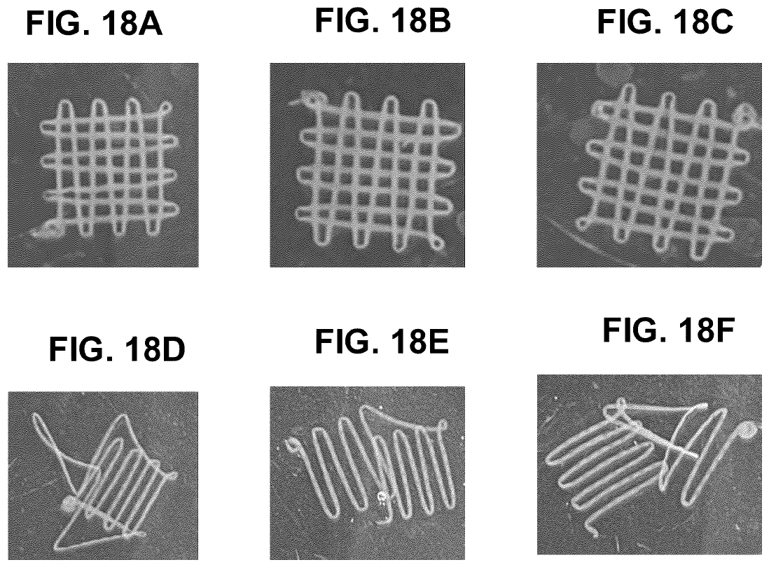

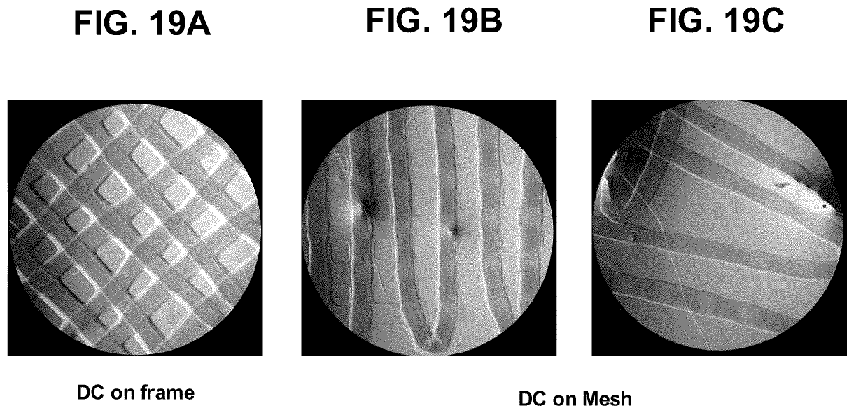

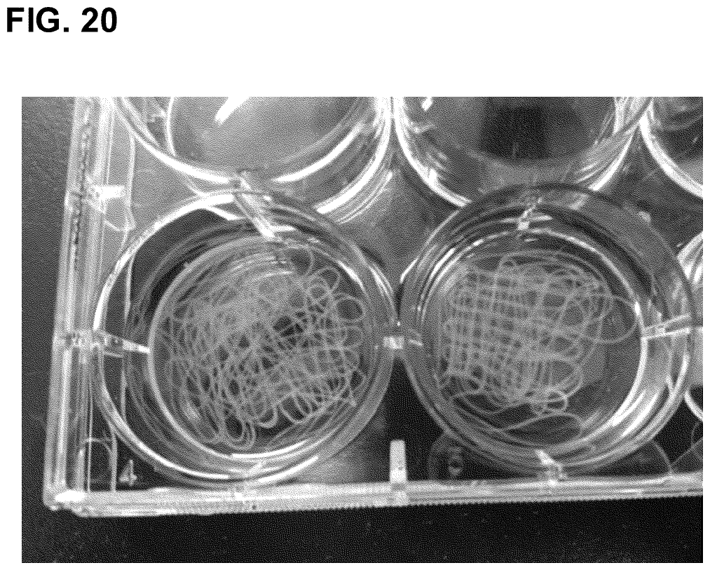

All three coated lattice structures printed and coated via the reliance on a frame as herein disclosed passed the stability test (FIG. 17, and FIGS. 18A-18C as compared to 18D-18F). All three coated lattice structures printed onto mesh in lieu of a frame failed the stability test, and microscopic images (FIGS. 19A-19C) reveal fibers in the first layer escaped from the coating layer (compare FIGS. PB-PC with that of FIG. 19A). FIG. 20 depicts images of other fiber structures printed onto mesh (but not coated), illustrating degraded stability. The fiber structures of FIG. 20 were created by inside-out crosslinking.

### Example 4. Double Dip Coating of a Bioprinted Fiber Structure

In this Example, the bioprinted lattice structures tested were 16×16 mm, and 2 layers thick and printed on a device comprising a vessel for containing the dip-coating solution that was coupled to a vacuum chuck. The coated bioprinted lattice structures included a core (1.5% SLG100 with cells or dye), a shell (2% SLG100), a first inner conformal coating (2% SLG100) and a second outer conformal coating (2% Zwit-20 Alginate). The lattice structures were printed via the use of a frame as herein disclosed. The inner and outer conformal coatings were added to the bioprinted lattice structure while coupled to the frame.

After printing of the 2-layer, 16×16 mm lattice structures, the structures were cross-linked with 95%/5% Ca/Ba in 15% polyethylene glycol (PEG) in pH-buffered dH₂O for 3 minutes. The cross-linking bath was removed by vacuuming and the lattice structures were rinsed with TSC saline from the buffer channel on the printhead. All solution was then removed by vacuuming and the lattice structures were raised 3 to 5 mm from the receiving surface.

A first conformal coating solution of 1 mL of 2% SLG100 was then pipetted into the vessel to evenly coat the entire lattice structure, ensuring that all fibers were covered with the solution from both the top and bottom. The lattice structure was incubated in the first conformal coating solution for 10 seconds after which the extra solution was removed by vacuum from the vessel.

A second conformal coating solution of 1 mL of Zwit-20 alginate was then pipetted into the vessel to evenly coat the entire lattice structure, ensuring that all fibers were covered with the solution from both the top and bottom. The lattice structure was incubated in the second conformal coating solution for 35 seconds after which the extra solution was removed by vacuum from the vessel.

The lattice structure was then transferred to a 95%/5% Ca/Ba bath and allowed to cross-link for 3 minutes after which the lattice structure was rinsed with saline.

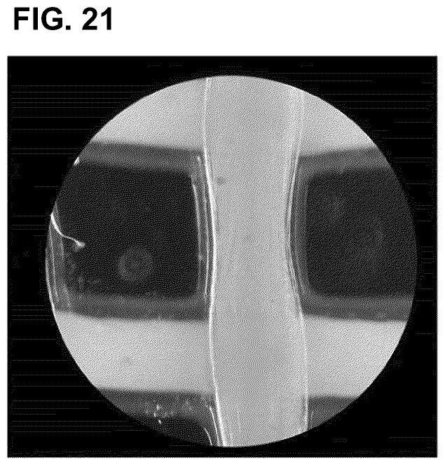

Photographs of the lattice structure with the inner and outer conformal coating were taken and are shown in FIG. 21. The diameter of the entire vertical fiber with both conformal coatings as depicted in FIG. 21 was 1.004 mm, the inner conformal coating was between 40.8 μm (left side, midpoint) and 50 μm (right side, midpoint) and the outer conformal coating was between 88.1 μm (left side, midpoint) and 90 μm (right side, midpoint).

### Example 5. Compartmentalization of the Core-Shell Fiber

Compartmentalization of the core-shell fiber was tested by printing a 11×11 mm lattice structure with or without cell aggregates in the core. The core material was 1.5% SLG100 with or without cells (cell density, 7×10⁷ cells/mL, cell number: 3×10⁶ cells) with a 2% SLG100 shell and 2% SLG100 conformal coating produced by dip-coating.

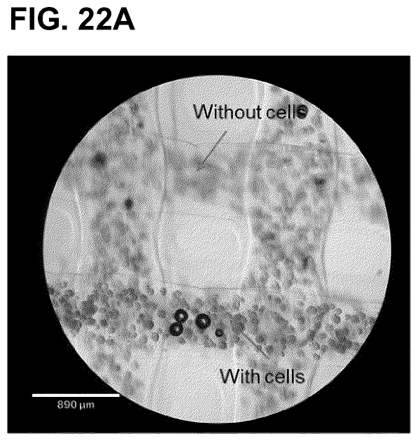

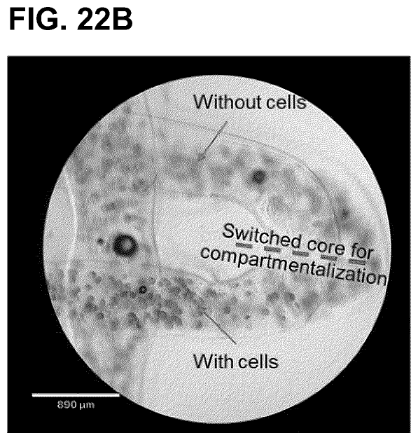

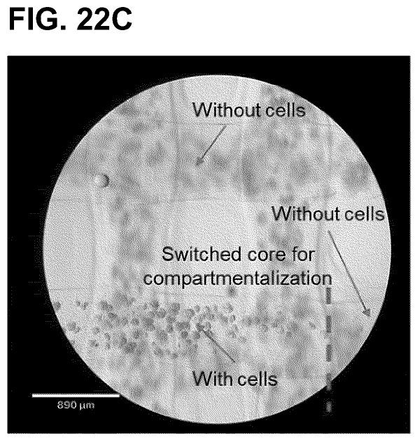

Images of the lattice structure are provided in FIGS. 22A-22C which show A) a top horizontal fiber without cells and a bottom horizontal fiber with cell aggregates, B) switching of the core for compartmentalization (dotted line) where the top portion of the loop is without cells and the bottom portion of the loop has cell aggregates after core-switching, and C) a top horizontal fiber without cells, switching for compartmentalization (dotted line) and a lower horizontal fiber with cell aggregates. These figures demonstrate that compartmentalized lattice structures can be produced such that portions of the lattice structure can be with or without cells.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. An implantable composition/medical device for the treatment of diabetes, comprising a multilayer lattice structure comprising a continuously bioprinted core/shell fiber encapsulating a plurality of pancreatic islet cells, and at least one coating surrounding said multilayer lattice structure, wherein the multilayer lattice structure has an infill density of between about 10% and about 90%, or between about 20% and about 80%, or between about 30% and about 70%, or between about 40% and about 60%.

2. The composition/device of claim 1, wherein said lattice structure has an infill density of about 30%, about 40%, about 50%, about 60%, or about 70%, or about 80%.; and preferably between about 50% and about 70%, or between about 55% and about 65%, or about 60%.

3. The composition/device of claim 1, wherein the multilayer lattice structure comprises at least one conformal coating.

4. The composition/device of claim 3, wherein the multilayer lattice structure comprises a first inner coating and a second outer coating; preferably wherein the first inner coating comprises a hydrogel having a material strength greater than that of the second outer coating.

5. The composition/device of claim 1, wherein the continuously bioprinted core/shell fiber comprises a solid core and at least one shell, optionally wherein the solid core has a material strength less than that of the shell.

6. The composition/device of claim 5, wherein the coating comprises a hydrogel having a material strength less than both the core and the at least one shell of the fiber.

7. The composition/device of claim 5, wherein the solid core, the at least one shell, and the coating comprise the same hydrogel material; preferably wherein the hydrogel material is alginate.

8. The composition/device of claim 7, wherein the solid core, the at least one shell, and/or the coating comprises a chemically modified alginate.

9. The composition/device of claim 7, wherein the solid core comprises between about 1.2 to about 1.8% alginate, preferably about 1.5% alginate.

10. The composition/device of claim 7, wherein the at least one shell comprises between about 1.4% to about 3.0% alginate; preferably between about 1.5% to about 2.5% alginate; more preferably between about 1.8% and about 2.2% alginate.

11. The composition/device of claim 7, wherein the coating comprises between about 0.2% alginate to about 2% alginate, or between about 0.25% alginate to about 1.5% alginate; preferably between about 0.3% to about 1.0% alginate; more preferably between about 0.4% to about 0.8% alginate.

12. The composition/device of any one of the preceding claims, wherein the pancreatic islet cells are human pancreatic islet cells, optionally wherein the pancreatic islet cells comprise re-aggregated islets.

13. The composition/device of any one of the preceding claims, wherein the lattice structure comprises at least two, three, four, or five layers formed by the continuous fiber, preferably wherein the lattice structure comprises two layers or three layers or four layers, more preferably wherein the lattice structure comprises four layers.

14. The composition/device of any one of the preceding claims, wherein a diameter of the continuous fiber is between about 0.2-2.0 mm, or between about 0.5-1.5 mm, between about 0.5-0.9 mm, or between about 900 μm to about 1200 μm, preferably wherein the diameter is between about 950 μm to about 1100 μm.

15. The composition/device of claim 14, wherein the solid core has a diameter between about 500 μm and about 800 μm, preferably between about 600 μm and about 700 μm, more preferably about 650 μm.

16. The composition/device of claim 14 or 15, wherein the at least one shell has a thickness of between about 50 μm and about 125 μm, preferably between about 75 μm and about 100 μm.

17. The composition/device of any one of claims 13-15, wherein the coating has a thickness of between about 50 μm to about 125 μm, preferably between about 75 μm and about 100 μm.

18. The composition/device of any one of the preceding claims, wherein the solid core and/or the at least one shell is compartmentalized along the length of the fiber.

19. The composition/device of claim 1, wherein the plurality of pancreatic islet cells are encapsulated in the solid core.

20. The composition/device of claim 1, wherein the plurality of pancreatic islet cells are encapsulated in the at least one shell.

21. A method of delivering insulin to a subject in need thereof, the method comprising implanting the composition/medical device of any one of claims 1-20 into the subject.

22. A method of treating a diabetic subject, comprising implanting the composition/medical device of any one of claims 1-20 into the diabetic subject.

23. The method according to claim 21 or 22, wherein the subject is a human subject suffering from Type 1 diabetes.

24. The method according to claim 21 or 22, wherein the implanting is carried out via a laparoscopic procedure.

25. The method according to any one of claims 21-24, further comprising retrieving the composition/medical device from the subject after 1-12 months, 18 months, 24 months, 2 years, 3 years, 4 years, 6 years, 8 years, or 10 years following implantation of the composition/medical device.

26. The method according to claim 25, further comprising implanting another composition/medical device of any one of claims 1-20 into said subject following said retrieving step.

27. A method of fabricating the implantable composition/medical device of any one of claims 1-20, comprising:
providing a bioprinting system comprising a fabrication platform for supporting a continuously bioprinted fiber during printing, patterning, and/or processing, the fabrication platform comprising a frame defining a void and comprising a plurality of posts on opposing sides of the frame for securing and suspending the continuously bioprinted fiber during printing;
a print head comprising a plurality of microfluidic channels to selectively provide a respective plurality of materials to a dispensing orifice; a positioning unit for positioning the frame in three dimensional space with respect to the print head; and at least one dispensing means for dispensing the fiber from the dispensing orifice;
via the bioprinting system, dispensing the fiber around a plurality of said posts to form lattice structure comprising at least two, three, four, or five layers of the fiber; and
coating the lattice structure after printing is completed.

28. The method according to claim 27, wherein said fabrication platform is submerged in a cross-linker bath during dispensing of said continuously bioprinted fiber.

29. The method according to claim 27, wherein said fabrication platform comprising said lattice structure is submerged into a cross-linker bath following dispensing of said continuously bioprinted fiber.

30. The method according to claim 27, wherein said coating step comprises submerging the fabrication platform comprising the lattice structure in at least one coating solution.

31. The method according to claim 27, wherein said coating step comprises dispensing at least one coating solution onto the lattice structure via the dispensing orifice following dispensing of said continuously bioprinted fiber.
